(19) United States
(12) Patent Application Publication
(10) Pub. No.: US 2026/0327582 A1
(43) Pub. Date: Oct. 22, 2026

(54) ENGINEERED PROTEINS AND METHODS OF USE THEREOF

(71) Applicant: Pairwise Plants Services, Inc., Durham, NC (US)

(72) Inventors: David Gerhard Schwark, New Hill, NC (US); Joseph Matthew Watts, Cary, NC (US)

(21) Appl. No.: 19/643,937

(22) Filed: Apr. 10, 2026

Related U.S. Application Data
(60) Provisional application No. 63/787,134, filed on Apr. 11, 2025.

(51) Int. Cl. C12N 9/12; C07K 14/00; C12N 9/22; C12N 15/11; C12N 15/90

(52) U.S. Cl. CPC C12N 9/1276; C07K 14/001; C12N 9/226; C12N 15/11; C12N 15/907; C12Y 207/07049; C12N 2310/20

(57) ABSTRACT

Described herein are polypeptides and methods of use of such polypeptides. Also described herein are complexes, compositions, and systems including polypeptides of the present invention, each of which may be used for modifying and/or editing a target nucleic acid. A polypeptide of the present invention may be an enzyme that generates DNA (e.g., cDNA) from RNA.

### STATEMENT REGARDING ELECTRONIC FILING OF A SEQUENCE LISTING

A Sequence Listing in XML format, entitled 1499-148_ST26.xml, 1,067,370 bytes in size, generated on Apr. 8, 2026, and filed herewith, is hereby incorporated by reference in its entirety for its disclosures.

### FIELD

This invention relates to engineered proteins (e.g., engineered enzymes) and to methods of use of such proteins. The invention further relates to compositions and systems for modifying or editing a target nucleic acid.

### BACKGROUND OF THE INVENTION

Reverse Transcriptases (RTs) (also known as RNA-Dependent DNA polymerases) are enzymes that generate DNA (e.g., cDNA) from RNA. They typically polymerize from free 3′ ends of primers annealed to the RNA. These primers can be either DNA or RNA depending on the system. In retroviruses, for example, the first strand primer is typically a transfer RNA. However, DNA is often used as a primer for RTs in vitro and during second strand syntheses in lentiviruses.

RTs are widely used for in vitro generation of cDNA in laboratories interested in studying mRNA. In these situations, poly dT DNA is used as a reverse strand primer and random deoxy hexamers are often used as forward strand primers. The most commonly used RTs for this function are Moloney Murine Leukemia Virus Reverse Transcriptase (MMLV-RT or M-MuLV-RT), Avian Myeloblastosis Virus Reverse Transcriptase (AMV-RT), and occasionally Human Immunodeficiency Virus Reverse Transcriptase (HIV-RT). The most commonly used of these three is MMLV-RT.

The vast majority of RT engineering has focused on RTs that have long processivity and high temperature tolerance like MMLV-RT. The reason for high temperature optimization of RTs has to do with the process of generating cDNA. Large RNA secondary structures can block reverse transcription. High temperatures can be used to partially denature RNA to remove or reduce or weaken secondary structure which further enables or facilitates the formation of cDNA. Processivity is also critical to generating good cDNA libraries, with many mRNA molecules having more than a thousand nucleotides. However, new RTs are desired, particularly RTs that can be used in template editing applications such as REDRAW and PRIME.

### SUMMARY OF THE INVENTION

A first aspect of the present invention is directed to a polypeptide comprising an amino acid sequence having at least 70%, 75%, 80%, 85%, 90%, 95%, 96%, 97%, 98%, 99%, or more sequence identity to SEQ ID NO:258 when the amino acid sequence is optimally aligned thereto, wherein the amino acid sequence, when optimally aligned to SEQ ID NO:258, has: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; a D at an amino acid residue corresponding to amino acid residue 148 of SEQ ID NO:258; an A, N, D, R, C, E, Q, G, I, leucine (L), K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; an A, N, D, R, C, Q, G, H, I, L, K, M, F, P, S, T, W, Y, or V at an amino acid residue corresponding to amino acid residue 171 of SEQ ID NO:258; a D at an amino acid residue corresponding to amino acid residue 181 of SEQ ID NO:258; an R at an amino acid residue corresponding to amino acid residue 221 of SEQ ID NO:258; a G at an amino acid residue corresponding to amino acid residue 225 of SEQ ID NO:258; a V at an amino acid residue corresponding to amino acid residue 230 of SEQ ID NO:258; an F at an amino acid residue corresponding to amino acid residue 234 of SEQ ID NO:258; an R or S at an amino acid residue corresponding to amino acid residue 236 of SEQ ID NO:258; an H at an amino acid residue corresponding to amino acid residue 237 of SEQ ID NO:258; a G at an amino acid residue corresponding to amino acid residue 241 of SEQ ID NO:258; an H at an amino acid residue corresponding to amino acid residue 252 of SEQ ID NO:258; a V at an amino acid residue corresponding to amino acid residue 256 of SEQ ID NO:258; a D at an amino acid residue corresponding to amino acid residue 275 of SEQ ID NO:258; an H at an amino acid residue corresponding to amino acid residue 287 of SEQ ID NO:258; an R at an amino acid residue corresponding to amino acid residue 289 of SEQ ID NO:258; an F at an amino acid residue corresponding to amino acid residue 325 of SEQ ID NO:258; an R at an amino acid residue corresponding to amino acid residue 327 of SEQ ID NO:258; an A at an amino acid residue corresponding to amino acid residue 332 of SEQ ID NO:258; and/or a Y at an amino acid residue corresponding to amino acid residue 335 of SEQ ID NO:258. In some embodiments, the polypeptide generates DNA (e.g., cDNA) from RNA. In some embodiments, the polypeptide further comprises an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and/or a W at an amino acid residue corresponding to amino acid residue 603 of SEQ ID NO:258. The polypeptide may generate (e.g., polymerize) the DNA from one end (e.g., the 3′end) of a DNA and/or RNA primer. In some embodiments, the polypeptide has activity as an RNA-dependent DNA polymerase.

A further aspect of the present invention is directed to a polypeptide that has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524.

An additional aspect of the present invention is directed to a nucleic acid molecule encoding a polypeptide of the present invention. In some embodiments, the polypeptide has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524. In some embodiments, a nucleic acid molecule of the present invention has at least about 70%, 75%, 80%, 85%, 90%, 95%, 96%, 97%, 98%, 99%, or more sequence identity to one or more of SEQ ID NOs: 340-347, 349-359, 379-394, 396-401, 403-405, 407, and 409.

Another aspect of the present invention is directed to a complex comprising a CRISPR-Cas effector protein; a polypeptide of the present invention; and an extended guide nucleic acid. In some embodiments, the polypeptide has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524.

Another aspect of the present invention is an expression cassette codon optimized for expression in an organism, comprising a polynucleotide encoding a promoter sequence, and a polynucleotide encoding a polypeptide of the present invention, which may be codon-optimized for expression in the organism. In some embodiments, an expression cassette of the present invention comprises a polynucleotide encoding a promoter sequence, and a polynucleotide that has at least about 70%, 75%, 80%, 85%, 90%, 95%, 96%, 97%, 98%, 99%, or 100% sequence identity to one or more of SEQ ID NOs: 340-347, 349-359, 379-394, 396-401, 403-405, 407, and 409, which may be codon-optimized for expression in an organism.

A further aspect of the present invention is directed to a method of modifying a target nucleic acid in a cell, the method comprising introducing an expression cassette and/or vector of the present invention into the cell, thereby modifying the target nucleic acid in the cell.

An additional aspect of the present invention is directed to a method for producing a polypeptide of the present invention, the method comprising culturing a cell or group of cells that have been transformed with a nucleic acid encoding said polypeptide; and isolating the polypeptide, thereby producing the polypeptide.

Another aspect of the present invention is directed to a method for performing reverse transcription, the method comprising contacting a target nucleic acid with a polypeptide of the present invention, wherein the polypeptide reverse transcribes the target nucleic acid to provide a DNA (e.g., a cDNA). In some embodiments, the polypeptide has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524.

A further aspect of the present invention is directed to a method of modifying a target nucleic acid, the method comprising contacting the target nucleic acid with a CRISPR-Cas effector protein; a polypeptide of the present invention; and an extended guide nucleic acid, thereby modifying the target nucleic acid. In some embodiments, the polypeptide has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a graph showing the normalized precise editing percentage for MMLV-RT (5M) reverse transcriptase proteins mutated at amino acid residue 66 targeting two sites in the DNMT1-001 (PWsp143 and PWsp137) locus and one site in the RNF2 (PWsp454) locus.

FIG. 2 is a graph showing the normalized precise editing percentage for MMLV-RT (5M) reverse transcriptase proteins mutated at amino acid residue 161 targeting two sites in the DNMT1-001 (PWsp143 and PWsp137) locus and one site in the RNF2 (PWsp454) locus.

FIG. 3 is a graph showing the normalized precise editing percentage for MMLV-RT (5M) reverse transcriptase proteins mutated at amino acid residue 168 targeting two sites in the DNMT1-001 (PWsp143 and PWsp137) locus and one site in the RNF2 (PWsp454) locus.

FIG. 4 is a graph showing the normalized precise editing percentage for MMLV-RT (5M) reverse transcriptase proteins mutated at amino acid residue 171 targeting two sites in the DNMT1-001 (PWsp143 and PWsp137) locus and one site in the RNF2 (PWsp454) locus.

FIG. 5 is a graph showing the normalized precise editing percentage for MMLV-RT (5M) reverse transcriptase proteins mutated at amino acid residue 139 targeting two sites in the DNMT1-001 (PWsp143 and PWsp137) locus and one site in the RNF2 (PWsp454) locus.

FIG. 6 is a graph showing the normalized precise editing percentage for MMLV-RT (5M) reverse transcriptase proteins mutated at amino acid residue 223 targeting two sites in the DNMT1-001 (PWsp143 and PWsp137) locus and one site in the RNF2 (PWsp454) locus.

FIG. 7 is a graph showing the precise editing percentage for mutant MMLV-RT (5M) reverse transcriptase proteins according to some embodiments of the present invention targeting the first DNMT1-001 (PWsp143) locus.

FIG. 8 is a graph showing the precise editing percentage for mutant MMLV-RT (5M) reverse transcriptase proteins according to some embodiments of the present invention targeting the second DNMT1-001 (PWsp137) locus.

FIG. 9 is a graph showing the insertion-deletion (INDEL) percentages for mutant MMLV-RT (5M) reverse transcriptase proteins according to some embodiments of the present invention targeting the first DNMT1-001 (PWsp143) locus.

FIG. 10 is a graph showing the INDEL percentages for mutant MMLV-RT (5M) reverse transcriptase proteins according to some embodiments of the present invention targeting the second DNMT1-001 (PWsp137) locus.

FIG. 11 is a graph showing the ratio of precise editing to INDEL formation for mutant MMLV-RT (5M) reverse transcriptase proteins according to some embodiments of the present invention targeting the first DNMT1-001 (PWsp143) locus.

FIG. 12 is a graph showing the ratio of precise editing to INDEL formation for mutant MMLV-RT (5M) reverse transcriptase proteins according to some embodiments of the present invention targeting the second DNMT1-001 (PWsp137) locus.

FIG. 13 is a graph showing the precise editing power for various mutants according to some embodiments of the present invention.

FIG. 14 is a graph showing the deletion power for various mutants according to some embodiments of the present invention.

FIG. 15 is a graph showing the precise editing power for various mutants according to some embodiments of the present invention.

FIG. 16 is a graph showing the deletion power for various mutants according to some embodiments of the present invention.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “heterologous nucleotide sequence” or a “recombinant nucleotide sequence” is a nucleotide sequence not naturally associated with a host cell into which it is introduced, including non-naturally occurring multiple copies of a naturally occurring nucleotide sequence.

A “native” or “wild-type” nucleic acid, nucleotide sequence, polypeptide or amino acid sequence refers to a naturally occurring or endogenous nucleic acid, nucleotide sequence, polypeptide or amino acid sequence. Thus, for example, a “native nucleic acid” is a nucleic acid that is naturally occurring in or endogenous to a reference organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “recombinant nucleic acid,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions).

A polynucleotide, gene, or polypeptide may be “isolated” by which is meant a nucleic acid or polypeptide that is substantially or essentially free from components normally found in association with the nucleic acid or polypeptide, respectively, in its natural state. In some embodiments, such components include other cellular material, culture medium from recombinant production, and/or various chemicals used in chemically synthesizing the nucleic acid or polypeptide.

The term “mutation” refers to point mutations (e.g., missense or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

As used herein, a “non-natural mutation” refers to a mutation that is generated through human intervention and differs from mutations found in the same gene or polypeptide that have occurred in nature (e.g., occurred naturally and not as a result of a modification made by a human).

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., “substantially complementary,” such as about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence or polypeptide (including a domain) will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of a nucleotide sequence or polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide. In some embodiments, a portion of a reference nucleotide sequence or polypeptide is about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or more of the full-length reference nucleotide sequence or polypeptide. Such a nucleic acid fragment or portion according to the invention may be, where appropriate, included in a larger polynucleotide of which it is a constituent. As an example, a repeat sequence of guide nucleic acid of this invention may comprise a portion of a wild-type CRISPR-Cas repeat sequence (e.g., a wild-type Type V CRISPR Cas repeat, e.g., a repeat from the CRISPR Cas system that includes, but is not limited to, Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c, and the like). Similarly, a portion of a polypeptide may be included in a larger polypeptide of which it is a constituent.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptides of this invention. “Orthologous” and “orthologs” as used herein, refers to homologous nucleotide sequences and/or amino acid sequences in different species that arose from a common ancestral gene during speciation. A homologue or ortholog of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which one or more test sequence(s) are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package® (Accelrys Inc., San Diego, CA) as well as web-based alignment programs such as Clustal Omega, EMBOSS Needle, EMBOSS Stretcher, EMBOSS Water, LALIGN, GGSEARCH2SEQ, EMBOS Cons, Kalign, MAFFT, MUSCLE, and T-Coffee. In some embodiments, an “optimal alignment” of two sequences (e.g., two polypeptide sequences) is the highest scoring alignment, optionally from an alignment conducted by a tool such as the local homology algorithm of Smith and Waterman, the homology alignment algorithm of Needleman and Wunsch, the search for similarity method of Pearson and Lipman, GAP, BESTFIT, FASTA, and TFASTA available as part of the GCG® Wisconsin Package® (Accelrys Inc., San Diego, CA), Clustal Omega, EMBOSS Needle, EMBOSS Stretcher, EMBOSS Water, LALIGN, GGSEARCH2SEQ, EMBOS Cons, Kalign, MAFFT, MUSCLE, and/or T-Coffee. In some embodiments, an “optimal alignment” of two sequences (e.g., two polypeptide sequences) is the alignment that provides the highest percent sequence identity, optionally allowing for one or more gap(s) to be introduced into one or both sequences. An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” and/or optimal alignment may be determined using Basic Local Alignment Search Tool (BLAST) provided by the National Center for Biotechnology Information such as BLASTX, for translated nucleotide sequences, BLASTN for polynucleotide sequences, and BLASTP for polypeptide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention can be codon optimized for expression. In some embodiments, a polynucleotide, nucleic acid construct, expression cassette, and/or vector of the present invention (e.g., that comprises/encodes a polypeptide of the present invention (e.g., an engineered protein), a nucleic acid binding polypeptide (e.g., a DNA binding polypeptide such as a sequence-specific DNA binding domain from a polynucleotide-guided endonuclease, a zinc finger nuclease, a transcription activator-like effector nuclease (TALEN), an Argonaute protein, and/or a CRISPR-Cas effector protein), a guide nucleic acid, and/or a reverse transcriptase) may be codon optimized for expression in an organism (e.g., an animal such as a human, a plant, a fungus, an archaeon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9% or 100%) identity or more to the reference nucleic acid constructs, polynucleotides, expression cassettes, and/or vectors but which have not been codon optimized.

In any of the embodiments described herein, a polynucleotide or nucleic acid construct of the invention may be operatively associated with a variety of promoters and/or other regulatory elements for expression in an organism or cell thereof (e.g., a mammal and/or a mammalian cell, a plant and/or a cell of a plant, etc.). Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” or “fused” in reference to polypeptides, refers to the covalent attachment of one polypeptide to another. A polypeptide may be linked or fused to another polypeptide (e.g., at the N-terminus or the C-terminus) directly (e.g., via a peptide bond) or through a linker (e.g., a peptide linker). Two polypeptides being directly fused (e.g., a direct linkage) refers to the covalent attachment of one amino acid residue of a first polypeptide of the two polypeptides to an amino acid residue of a second polypeptide of the two polypeptides without an intervening element between the two amino acid residues. For example, first and second polypeptides may be directly linked via a peptide bond between the first and second polypeptides without an intervening element (e.g., a linker) between the first and second polypeptides. Two polypeptides being indirectly fused (e.g., an indirect linkage) refers to an intervening element (e.g., a linker such as a peptide linker) that is present between the two polypeptides and is covalently attached to each, optionally the intervening element may attach one end of a first polypeptide of the two polypeptides to an end of the second polypeptide of the two polypeptides.

A “fusion protein” as used herein refers to two or more polypeptides that are covalently attached (e.g., directly or indirectly) so that they are transcribed and translated as a single unit and thereby produce a single polypeptide comprising the two or more polypeptides. In some embodiments, the two or more polypeptides may naturally be encoded by separate genes, but, in the form of a fusion protein, are encoded by a single gene. The term “linker” is art-recognized and refers to a chemical group, or a molecule linking two molecules or moieties, e.g., linking two polypeptides or domains of a fusion protein, such as, for example, a CRISPR-Cas effector protein and a peptide tag and/or a polypeptide of interest. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide (e.g., a peptide linker).

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length)). In some embodiments, a peptide linker may be a GS linker. In some embodiments, the peptide linker is a GS linker having 2, 3, or 4 amino acid residues, optionally 2 or 4 amino acid residues. In some embodiments, the peptide linker has one of the amino acid sequences of SEQ ID NOs: 1-36. In some embodiments, the peptide linker may comprise an amino acid sequence of CA, CF, (GGS)ₙ, GS, SG, GSSG (SEQ ID NO: 31), GSSGSS (SEQ ID NO:32), GSSGSSGS (SEQ ID NO:33), (GSS)ₙ (SEQ ID NO: 34), (GSS)ₙGS (SEQ ID NO:35), S(GGS)ₙ (SEQ ID NO:25), SGGS (SEQ ID NO:26), (GSS)ₙG (SEQ ID NO:36), or (GGGGS)ₙ (SEQ ID NO:27), wherein n is an integer of 1-20 (e.g., 1, 2, 3, 4, 5, 6, 7, 8, 9, 10, 11, 12, 13, 14, 15, 16, 17, 18, 19, or 20). In some embodiments, the peptide linker may comprise the amino acid sequence: SGGSGGSGGS (SEQ ID NO:28). In some embodiments, the peptide linker may comprise the amino acid sequence: SGSETPGTSESATPES (SEQ ID NO:29), also referred to as the XTEN linker. In some embodiments, the peptide linker may comprise the amino acid sequence: SGGSSGGSSGSETPGTSESATPESSGGSSGGS (SEQ ID NO:30), also referred to as the GS-XTEN-GS linker.

As used herein, the term “linked,” or “fused” in reference to polynucleotides, refers to the covalent attachment of one polynucleotide to another polynucleotide. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a direct covalent linkage or through one or more linking nucleotides. In some embodiments, a polynucleotide motif of a certain structure may be inserted within another polynucleotide sequence (e.g., extension of the hairpin structure in guide RNA). In some embodiments, the linking nucleotides may be naturally occurring nucleotides. In some embodiments, the linking nucleotides may be non-naturally occurring nucleotides. Two polynucleotides being directly fused (e.g., a direct linkage) refers to the covalent attachment of one nucleotide of a first polynucleotide of the two polynucleotides to a nucleotide of a second polynucleotide of the two polynucleotides without an intervening element between the two polynucleotides. For example, first and second polynucleotides may be directly linked via a phosphodiester bond between the first and second polynucleotides without an intervening element (e.g., a linker) between the first and second polynucleotides. Two polynucleotides being indirectly fused (e.g., an indirect linkage) refers to an intervening element (e.g., a linker such as a polynucleotide linker) that is present between the two polynucleotides and is covalently attached to each, optionally the intervening element attaches one end of a first polynucleotide of the two polynucleotides to an end of the second polynucleotide of the two polynucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NO:37 or SEQ ID NO:38).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdcal are inducible promoters. Pnr is induced by nitrate and repressed by ammonium (Li et al. Gene 403:132-142 (2007)) and Pdcal is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79:87-94), maize (Christensen et al., 1989. Plant Molec. Biol. 12:619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the European patent publication EP0342926. The ubiquitin promoter is suitable for the expression of the nucleotide sequences of the invention in transgenic plants, especially monocotyledons. Further, the promoter expression cassettes described by McElroy et al. (Mol. Gen. Genet. 231:150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, incorporated by reference herein for its disclosure of promoters. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); European patent EP 0452269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60 (6): 485-495 (2017)), LAT52 and LAT59 from tomato (Twell et al. Development 109(3):705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO 1999/042587).

Additional examples of plant tissue-specific/tissue preferred promoters include, but are not limited to, the root hair-specific cis-elements (RHEs) (KIM ET AL. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37 (8): 1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron. In some embodiments, an intron useful with this invention comprises a polynucleotide sequence with at least about 70%, 75%, 80%, 85%, 90%, 95%, 96%, 97%, 98%, 99%, or 100% sequence identity to SEQ ID NO:512. In some embodiments, an intron useful with this invention comprises a polynucleotide having the sequence of SEQ ID NO:512.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

An “editing system” as used herein refers to any site-specific (e.g., sequence-specific) nucleic acid editing system now known or later developed, which system can introduce a modification (e.g., a mutation) in a nucleic acid in target specific manner. For example, an editing system (e.g., a site- and/or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which may comprise one or more polypeptide(s) and/or one or more polynucleotide(s) that when present and/or expressed together (e.g., as a system) in a composition and/or cell can modify (e.g., mutate) a target nucleic acid in a sequence specific manner. In some embodiments, an editing system (e.g., a site- and/or sequence-specific editing system) can comprise one or more polynucleotide(s) and/or one or more polypeptide(s), including but not limited to a nucleic acid binding polypeptide (e.g., a DNA binding domain), a nuclease, another polypeptide, and/or a polynucleotide. In some embodiments, a CRISPR-Cas editing system is provided and/or is used that comprises a polypeptide of the present invention.

In some embodiments, an editing system comprises one or more sequence-specific nucleic acid binding polypeptide(s) (e.g., a DNA binding domain) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system comprises one or more cleavage polypeptide(s) (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN).

A “nucleic acid binding polypeptide” as used herein refers to a polypeptide or domain that binds and/or is capable of binding a nucleic acid (e.g., a target nucleic acid). A DNA binding domain is an exemplary nucleic acid binding polypeptide and may be a site- and/or sequence-specific nucleic acid binding domain. In some embodiments, a nucleic acid binding polypeptide may be a sequence-specific nucleic acid binding polypeptide such as, but not limited to, a sequence-specific binding domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage domain (e.g., a nuclease domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, a nucleic acid binding polypeptide comprises one or more additional domains such as BRCA2 Exon27 (Brex27) comprising the amino acid sequence of SEQ ID NO:511. In some embodiments, the nucleic acid binding polypeptide associates with and/or is capable of associating with (e.g., forms a complex with) with one or more nucleic acid molecule(s) (e.g., forms a complex with a guide nucleic acid as described herein), which may direct and/or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein.

In some embodiments, an editing system comprises or is a ribonucleoprotein such as an assembled ribonucleoprotein complex (e.g., a ribonucleoprotein that comprises a CRISPR-Cas effector protein, a guide nucleic acid, and optionally a reverse transcriptase). In some embodiments, a ribonucleoprotein of an editing system may be assembled together (e.g., a pre-assembled ribonucleoprotein including a CRISPR-Cas effector protein, a guide nucleic acid, and optionally a reverse transcriptase) such as when contacted to a target nucleic acid or when introduced into a cell (e.g., a mammalian cell or a plant cell) (e.g., at the time of contacting the components of the ribonucleoprotein to a target nucleic acid and/or at the time of introducing the components of the ribonucleoprotein into a cell). In some embodiments, a ribonucleoprotein of an editing system may assemble into a complex (e.g., a non-covalently bound complex) while a portion of the ribonucleoprotein is contacting a target nucleic acid and/or may assemble after and/or during introduction into a plant cell. In some embodiments, a ribonucleoprotein of an editing system may be contacted to a target nucleic acid and/or may be introduced into a plant cell. In some embodiments, an editing system may be assembled (e.g., into a non-covalently bound complex) when introduced into a plant cell. In some embodiments, a ribonucleoprotein may comprise a polypeptide of the present invention, a guide nucleic acid, and optionally a reverse transcriptase.

In some embodiments, an editing system of the present invention comprises a reverse transcriptase, which may be a polypeptide of the present invention, an extended guide nucleic acid, and a CRISPR-Cas effector protein, e.g., a Type II CRISPR-Cas effector protein or Type V CRISPR-Cas effector protein. In some embodiments, the Type V CRISPR-Cas effector protein or Type II CRISPR-Cas effector protein, the reverse transcriptase, and the extended guide nucleic acid may form a complex or may be comprised in a complex that is capable of interacting with a target nucleic acid.

In some embodiments, a guide nucleic acid further comprises a reverse transcriptase template and may be referred to as an extended guide nucleic acid. An “extended guide nucleic acid” as used herein is a guide nucleic acid as described herein that further comprises a reverse transcriptase template (RTT) and/or a primer binding site (PBS). In some embodiments, an extended guide nucleic acid is an engineered prime editing guide RNA (pegRNA). An extended guide nucleic acid may be a targeted allele guide RNA (tagRNA) or a stabilized targeted allele guide RNA (stagRNA). A “tagRNA” as used herein refers to an extended guide nucleic acid that comprises a PBS and a RTT and has target strand complementarity. A “stagRNA” as used herein refers to a tagRNA that comprises a stabilization motif. A stabilization motif may be present at the 3′ and/or 5′ end of a tagRNA. In some embodiments, a stabilization motif is present at the 3′ end of a tagRNA. Exemplary stabilization motifs include, but are not limited to, recruiting motifs, RNA hairpins, pseudoknot sequences, and/or PP7 motifs (e.g., a PP7 RNA hairpin sequence). In some embodiments, a stagRNA is a tagRNA that comprises a PP7 RNA hairpin sequence. In some embodiments, a CRISPR-Cas effector protein (e.g., a Type II or Type V CRISPR-Cas effector protein), a reverse transcriptase, and an extended guide nucleic acid can form a complex or are comprised in a complex.

In some embodiments, an extended guide nucleic acid comprises an extended portion that includes a primer binding site and a reverse transcriptase template, wherein the reverse transcriptase template comprises the modification (e.g., edit) to be incorporated into a target nucleic acid. In some embodiments, an extended guide nucleic acid comprises, at its 3′ end, a primer binding site and a modification (e.g., an edit) to be incorporated into the target nucleic acid (e.g., a reverse transcriptase template). In some embodiments, an extended guide nucleic acid comprises: (1) a sequence that interacts (e.g., recruits and/or binds) with a CRISPR-Cas effector protein (e.g., a CRISPR-Cas nuclease), (2) a spacer having substantial complementary to a first site on a target nucleic acid (e.g., a CRISPR RNA (crRNA) (a first crRNA) and/or tracrRNA+crRNA (sgRNA)), and (3) a nucleic acid encoded repair template (e.g., an RNA encoded repair template) comprising a primer binding site and an RNA template (e.g., that encodes the modification to be incorporated into the target nucleic acid). In some embodiments, an extended guide nucleic acid (e.g., an extended guide RNA) may comprise, 5′-3′, a spacer sequence, a repeat sequence, and an extended portion, the extended portion comprising, 5′ to 3′, a reverse transcriptase template and a primer binding site. In some embodiments, an extended guide nucleic acid may comprise, 5′-3′, a spacer sequence, a repeat sequence and an extended portion, the extended portion comprising, 5′ to 3′, a primer binding site and a reverse transcriptase template. In some embodiments, an extended guide nucleic acid may comprise, 5′-3′, an extended portion, a spacer sequence, and a repeat sequence, wherein the extended portion comprises, 5′ to 3′, a reverse transcriptase template and a primer binding site. In some embodiments, an extended guide nucleic acid may comprise, 5′-3′, an extended portion, a spacer sequence, and a repeat sequence, wherein the extended portion comprises, 5′ to 3′, a primer binding site and a reverse transcriptase template.

According to some embodiments, an extended guide nucleic acid (e.g., a pegRNA) may have a structure and/or be designed as described in Anzalone et al., Nature, 2019 December; 576 (7785): 149-157. In some embodiments, an extended guide nucleic acid comprises a primer binding site (PBS) optionally having a sequence of 1, 2, 3, 4, or 5 to 6, 7, 8, 9, 10, 11, 12, 13, 14, or 15 nucleotides and a reverse transcriptase template (RT template) sequence optionally having a sequence of 65 nucleotides or more. In some embodiments, a PBS of an extended guide nucleic acid has a sequence of less than 15 nucleotides and has a sequence of 1, 2, 3, 4, 5, 6, 7, 8, 9, 10, 11, 12, 13, or 14 nucleotides (e.g., a sequence of 5 or 6 nucleotides in length). The RT template sequence may be after the PBS sequence in the 5′ to 3′ direction. In some embodiments, the RT template sequence of the extended guide nucleic acid has a length of greater than 65 nucleotides and may comprise about 50 or more nucleotides of heterology relative to the target site (e.g., target nucleic acid), followed by about 15 or more nucleotides of homology relative to the target site. In some embodiments, the RT template sequence of the extended guide nucleic acid is after the PBS sequence and the RT template sequence has a length of greater than 65 nucleotides with the sequence including more than 50 nucleotides of heterology relative to the target site, followed by more than 15 nucleotides of homology relative to the target site. Accordingly, in some embodiments, when the extended guide nucleic acid is reverse transcribed, the resulting newly transcribed sequence may hybridize and/or is configured to hybridize with the unnicked strand of the target site, which may thereby create a heteroduplex DNA with a large insertion into the newly synthesized strand. Upon repair of this mismatched DNA, the resultant repaired DNA may contain a large insertion (e.g., greater than 50 nucleotides) of DNA sequence. In some embodiments, the method may provide a large deletion (e.g., greater than 50 nucleotides) of DNA sequence. In some embodiments, the PBS and the 15 or more nucleotides of homology to the target site may comprise homology arms, which may serve to insert the heterology into the target site optionally using homology directed repair. The inserted DNA may correspond to any functional sequence of DNA such as, but not limited to: a functional transgene; a fragment of DNA that is inserted into a gene in a way that, when the gene is transcribed, would produce a hairpin RNA that is sufficient to silence homologous genes through RNAi; and/or one or more functional site-specific recombination sites, e.g. lox, frt, which could then be used in subsequent Cre or Flp mediated site-specific recombination processes. In some embodiments, an extended guide nucleic acid may be too large to produce using a PolIII promoter in vivo. In some embodiments, an extended guide nucleic acid may be operatively associated with and/or produced using a PolII promoter. In some embodiments, a DNA binding polypeptide (e.g., a DNA binding domain) and/or DNA endonuclease may have a structure and/or be designed as described in Anzalone et al., Nature, 2019 December; 576 (7785): 149-157. In some embodiments, a DNA binding domain and/or DNA endonuclease is a CRISPR Cas polypeptide such as a Cas9 nickase, a nicking variant of another CRISPR Cas polypeptide, or Cas12a.

In some embodiments, two extended guide nucleic acids (e.g., pegRNAs) may be used (e.g., an editing system may comprise two extended guide nucleic acids). One or both of the two extended guide nucleic acids may have a structure and/or be designed as described in Anzalone et al., Nature, 2019 December; 576 (7785): 149-157. The two extended guide nucleic acids may comprise a primer binding site (PBS) optionally having a sequence of 1, 2, 3, 4, or 5 to 6, 7, 8, 9, 10, 11, 12, 13, 14, or 15 nucleotides and a reverse transcriptase template (RT template) sequence optionally having a sequence of 50 nucleotides or more. The RT template sequences of the two extended guide nucleic acids may be complementary to each other and as such the polynucleotides that are respectively reverse transcribed from each the RT templates will be complementary to each other and will be able to hybridize with each other. This may allow for the intermediates that are produced by this system and/or method to join together two sections of DNA that are otherwise separated by more than 50 nucleotides, e.g., within a chromosome, or that are positioned on two separate pieces of DNA, e.g., on two different chromosomes. After repair of the intermediates, the resultant products may produce, depending on the design of the RT template, large deletions, large inversions, or inter-chromosomal recombinations. Since all of these products are produced by homology directed repair, the products may be predictably precise and/or reproducible. In some embodiments, a DNA binding polypeptide (e.g., a DNA binding domain) and/or DNA endonuclease may have a structure and/or be designed as described in Anzalone et al., Nature, 2019 December; 576 (7785): 149-157. In some embodiment, a DNA binding polypeptide and/or DNA endonuclease is a CRISPR Cas polypeptide such as a Cas9 nickase, a similar nicking variant of another CRISPR Cas polypeptide, or Cas12a. In some embodiments, a DNA binding polypeptide and/or DNA endonuclease is a Cas9 nuclease, a similar nuclease from another CRISPR Cas polypeptide, or Cas12a. Using a nuclease (rather than a nickase) may facilitate the intra- or interchromosomal recombination processes through single-strand annealing of the more than 50 nucleotide 3′ overhangs that would be produced at each of the two target sites corresponding to the two pegRNA target nucleic acids. In some embodiments, an editing system comprises one extended guide nucleic acid and a guide nucleic acid that is devoid of a reverse transcriptase template and/or primer binding site.

An extended guide nucleic acid may comprise a CRISPR nucleic acid (e.g., CRISPR RNA, CRISPR DNA, crRNA, crDNA) and/or a CRISPR nucleic acid and a tracr nucleic acid; and (b) an extended portion comprising a primer binding site and a reverse transcriptase template (RT template), wherein the RT template encodes a modification to be incorporated into the target nucleic acid. The CRISPR nucleic acid may be a Type II or Type V CRISPR nucleic acid and/or the tracr nucleic acid may be any tracr corresponding to the appropriate Type II or Type V CRISPR nucleic acid. In some embodiments, an extended guide nucleic acid comprises: (i) a Type V CRISPR nucleic acid or a Type II CRISPR nucleic acid (e.g., a Type II or Type V CRISPR RNA, Type II or Type V CRISPR DNA, Type II or Type V crRNA, or Type II or Type V crDNA) and/or a CRISPR nucleic acid and a tracr nucleic acid (e.g., a Type II or Type V tracrRNA, Type II or Type V tracrDNA); and (ii) an extended portion comprising a primer binding site and a reverse transcriptase template (RT template), wherein the Type V CRISPR nucleic acid or Type II CRISPR nucleic acid comprises a spacer that binds to a first strand (e.g., the target strand) of a target nucleic acid (e.g., the spacer is complementary to a portion of consecutive nucleotides in the first strand of the target nucleic acid) and the primer binding site binds to the first strand (e.g., target strand). In some embodiments, the extended portion can be fused to either the 5′ end or 3′ end of the CRISPR nucleic acid (e.g., from 5′ to 3′: repeat-spacer-extended portion or extended portion-repeat-spacer) and/or to the 5′ or 3′ end of the tracr nucleic acid. In some embodiments, the extended portion of an extended guide nucleic acid comprises, 5′ to 3′, an RT template (RTT) and a primer binding site (PBS) (e.g., 5′-crRNA-spacer-RTT (edit encoded)-PBS-3′) or comprises 5′ to 3′ a PBS and RTT, depending on the location of the extended portion relative to the CRISPR nucleic acid of the extended guide nucleic acid (e.g., 5′-crRNA-spacer-PBS-RTT (edit encoded)-3′). For example, in some embodiments, an extended portion of the extended guide nucleic acid may comprise, 5′ to 3′, an RT template and a primer binding site (when the extended guide is linked to the 3′ end of the CRISPR nucleic acid). In some embodiments, an extended portion of the extended guide may comprise, 5′ to 3′, a primer binding site and an RT template (when the extended guide is linked to the 5′ end of the CRISPR nucleic acid).

In some embodiments, a target nucleic acid is double stranded and comprises a first strand and a second strand and a primer binding site of an extended guide nucleic acid binds to the second strand (e.g., the non-target, top strand) of the target nucleic acid. In some embodiments, a target nucleic acid is double stranded and comprises a first strand and a second strand and a primer binding site of an extended guide nucleic acid binds to the first strand (e.g., binds to the target strand, optionally the same strand to which a CRISPR-Cas effector protein is recruited, bottom strand) of the target nucleic acid. In some embodiments, a target nucleic acid is double stranded and comprises a first strand and a second strand and the primer binding site of an extended guide nucleic acid binds to the second strand (e.g., the non-target strand, optionally the opposite strand from that to which the CRISPR-Cas effector protein is recruited) of the target nucleic acid. In some embodiments, a reverse transcriptase (RT) may add to the target strand of a target nucleic acid (e.g., the strand to which the spacer of the CRISPR nucleic acid of the extended guide nucleic acid is complementary and to which the CRISPR-Cas effector protein is recruited). In some embodiments, the reverse transcriptase (RT) adds to the non-target strand of a target nucleic acid (e.g., the strand that is complementary to the strand to which the spacer of the CRISPR nucleic acid is complementary and to which the CRISPR-Cas effector protein is recruited). Example methods and editing systems are described in International Patent Publication No. WO 2021/092130, International Patent Publication No. WO 2022/098993, and U.S. Patent Application Publication Nos. 2021/0147862, 2021/0130835, 2021/0147862, and 2022/0145334, each of which are incorporated herein by reference in their entirety.

The RT template of an extended guide nucleic acid may encode one or more modification(s) (e.g., edit(s)) to be incorporated into a target nucleic acid. The one or more modification(s) may be located in any position within an RT template (e.g., where the position location may be relative to the position of a protospacer adjacent motif (PAM) of the target nucleic acid). In some embodiments, an RT template has a modification at one or more positions from −1 to 23 (e.g., −1, 1, 2, 3, 4, 5, 6, 7, 8, 9, 10, 11, 12, 13, 14, 15, 16, 17, 18, 19, 20, 21, 22, or 23) relative to the position of a protospacer adjacent motif (PAM) (e.g., TTTG) in a target nucleic acid. In some embodiments, an RT template may comprise a modification located at nucleotide position −1, 1, 2, 3, 4, 5, 6, 7, 8, 9, 10, 11, 12, 13, 14, 15, 16, 17, 18, 19, 20, 21, 22 or 23. In some embodiments, an RT template may comprise a modification located at nucleotide position 4 to nucleotide position 17 (e.g., position 4, 5, 6, 7, 8, 9, 10, 11, 12, 13, 14, 15, 16, or 17) of the RT template relative to the position of a PAM of a target nucleic acid. In some embodiments, an RT template may comprise a modification located at nucleotide position 10 to nucleotide position 17 (e.g., position 10, 11, 12, 13, 14, 15, 16, or 17) of the RT template relative to the position of a PAM of a target nucleic acid. In some embodiments, an RT template may comprise a modification located at nucleotide position 12 to nucleotide position 15 (e.g., position 12, 13, 14, or 15) of the RT template relative to the position of a PAM of a target nucleic acid.

In some embodiments, an extended portion of an extended guide nucleic acid may comprise, 5′ to 3′, an RT template and a primer binding site (e.g., when the extended portion is linked to the 3′ end of a CRISPR nucleic acid). In some embodiments, an extended portion of an extended guide nucleic acid may comprise, 5′ to 3′, a primer binding site and an RT template (RTT) (e.g., when the extended portion is linked to the 5′ end of the CRISPR nucleic acid). In some embodiments, an RT template may have a length of about 1 nucleotide to about 100 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides, and any range or value therein), e.g., about 1 nucleotide to about 10 nucleotides, about 1 nucleotide to about 15 nucleotides, about 1 nucleotide to about 20 nucleotides, about 1 nucleotide to about 25 nucleotides, about 1 nucleotide to about 30 nucleotides, about 1 nucleotide to about 35, 36, 37, 38, 39 or 40 nucleotides, about 1 nucleotide to about 50 nucleotides, about 5 nucleotides to about 15 nucleotides, about 5 nucleotides to about 20 nucleotides, about 5 nucleotides to about 25 nucleotides, about 5 nucleotides to about 30 nucleotides, about 5 nucleotides to about 35, 36, 37, 38, 39 or 40 nucleotides, about 5 nucleotides to about 50 nucleotides, about 8 nucleotides to about 15 nucleotides, about 8 nucleotide to about 20 nucleotides, about 8 nucleotide to about 25 nucleotides, about 8 nucleotide to about 30 nucleotides, about 8 nucleotide to about 35, 36, 37, 38, 39 or 40 nucleotides, about 8 nucleotide to about 50 nucleotides in length, about 8 nucleotides to about 100 nucleotides, about 10 nucleotide to about 15 nucleotides, about 10 nucleotide to about 20 nucleotides, about 10 nucleotide to about 25 nucleotides, about 10 nucleotide to about 30 nucleotides, about 10 nucleotide to about 36 nucleotides, about 10 nucleotide to about 40 nucleotides, about 10 nucleotide to about 50 nucleotides, about 10 nucleotides to about 100 nucleotides in length and any range or value therein. In some embodiments, the length of an RT template may be at least 8 nucleotides, optionally about 8 nucleotides to about 100 nucleotides. In some embodiments, the length of an RT template is 36, 37, 38, 39 or 40 nucleotides or less (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length, or any value or range therein (e.g., about 1, 2, 3, 4, 5, 6, 7, 8, 9, 10, 11, 12, 13, 14, or 15 nucleotides in length to about 16, 17, 18, 19, 20, 21, 22, 23, 24, 25, 26, 27, 28, 29, 30, 31, 32, 33, 34, 35, 36, 37, 38, 39, or 40 nucleotides in length). In some embodiments, the length of an RT template may be at least 30 nucleotides, optionally about 30, 31, 32, 33, 34, 35, 36, 37, 38, 39, or 40 nucleotides in length to about to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range or value therein. In some embodiments, the length of an RT template may be about 36, 40, 44, 47, 50, 52, 55, 63, 72 or 74 nucleotides. Within the length of the RTT one or more modification(s) may be present. The one or more modification(s) may be located anywhere within the RTT, wherein the position of the modification may be described relative to the position of a protospacer adjacent motif (PAM) of a target nucleic acid. In some embodiments, an RT template may comprise a modification located at nucleotide position-1, 1, 2, 3, 4, 5, 6, 7, 8, 9, 10, 11, 12, 13, 14, 15, 16, 17, 18, 19, 20, 21, 22 or 23. In some embodiments, an RT template may comprise a modification located at nucleotide position 4 to nucleotide position 17 (e.g., position 4, 5, 6, 7, 8, 9, 10, 11, 12, 13, 14, 15, 16, or 17) of the RT template relative to the position of a protospacer adjacent motif (PAM) of a target nucleic acid. In some embodiments, an RT template may comprise a modification located at nucleotide position 10 to nucleotide position 17 (e.g., position 10, 11, 12, 13, 14, 15, 16, or 17) of the RT template relative to the position of a protospacer adjacent motif (PAM) of a target nucleic acid. In some embodiments, an RT template may comprise a modification located at nucleotide position 12 to nucleotide position 15 (e.g., position 12, 13, 14, or 15) of the RT template relative to the position of a protospacer adjacent motif (PAM) of a target nucleic acid.

As used herein, a “primer binding site” (PBS) of an extended portion of an extended guide nucleic acid (e.g., a tagRNA) refers to a sequence of consecutive nucleotides that can bind to a region or “primer” on a target nucleic acid, e.g., is complementary to the target nucleic acid primer. As an example, a CRISPR Cas effector protein (e.g., a Type II or Type V, e.g., Cas 9 or Casl2a) may nick/cut the DNA and the 3′ end of the cut DNA acts as a primer for the PBS portion of the extended guide nucleic acid. The PBS may be complementary to the 3′ end of a strand of the target nucleic acid and may bind and/or may be configured to bind to either the target strand or non-target strand. A primer binding site can be fully complementary to the primer or it may be substantially complementary (e.g., at least 70% complementary (e.g., 70% or about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9% or more)) to the primer of a target nucleic acid. In some embodiments, the length of a primer binding site of an extended portion may be about 1 nucleotide to about 100 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides, or any value or range therein), or about 4 nucleotide to about 85 nucleotides, about 10 nucleotide to about 80 nucleotides, about 20 nucleotide to about 80 nucleotides, about 25 nucleotides to about 80 nucleotides about 30 nucleotide to about 80 nucleotides, about 40 nucleotide to about 80 nucleotides, about 45 nucleotide to about 80 nucleotides, about 45 nucleotide to about 75 nucleotides, or about 45 nucleotide to about 60 nucleotides, or any range or value therein. In some embodiments, the length of a PBS may be at least 30 nucleotides, optionally about 30, 31, 32, 33, 34, 35, 36, 37, 38, 39, or 40 nucleotides to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range or value therein. In some embodiments, the length of a PBS may be about 8, 16, 24, 32, 40, 48, 56, 64, 72, or 80 nucleotides.

In some embodiments, an RTT may have a length of about 35 nucleotides to about 75 nucleotides and a PBS may have a length of about 30 nucleotides to about 80 nucleotides, optionally wherein the PBS may comprise a length of about 8, 16, 24, 32, 40, 48, 56, 64, 72, or 80 nucleotides and the RTT may comprise a length of about 36, 40, 44, 47, 50, 52, 55, 63, 72 or 74 nucleotides, or any combination thereof of the RTT length and/or PBS length.

In some embodiments, an extended portion of an extended guide nucleic acid may be fused to either the 5′ end or 3′ end of a Type II or a Type V CRISPR nucleic acid (e.g., 5′ to 3′: repeat-spacer-extended portion, or extended portion-repeat-spacer) and/or to the 5′ or 3′ end of the tracr nucleic acid. In some embodiments, when an extended portion is located 5′ of the crRNA, a Type V CRISPR-Cas effector protein is modified to reduce (or eliminate) self-processing RNAse activity.

In some embodiments, the extended portion of an extended guide nucleic acid may be linked to the Type II or Type V CRISPR nucleic acid and/or the Type II or Type V tracrRNA via a linker. In some embodiments, a linker have a length of about 1 to about 100 nucleotides or more (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length, and any range therein (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, about 40 to about 100, about 50 to about 100, or about 2, 3, 4, 5, 6, 7, 8, 9, 10, 11, 12, 13, 14, 15, 16, 17, 18, 19, 20, 21, 22, 23, 24, 25 nucleotides to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length (e.g., about 105, 110, 115, 120, 130, 140 150 or more nucleotides in length).

A guide nucleic acid and/or an extended guide nucleic acid may comprise one or more recruiting motifs as described herein, which may be linked to the 5′ end and/or the 3′ end of the guide nucleic acid and/or it may be inserted into the guide nucleic acid (e.g., within a hairpin loop of the guide nucleic acid). In some embodiments, an extended guide nucleic acid may be linked to an RNA recruiting motif. An extended guide nucleic acid and/or guide nucleic acid may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion of an extended guide nucleic acid.

In some embodiments, an editing system comprises an extended guide nucleic acid that is linked to an RNA recruiting motif and a reverse transcriptase that is a reverse transcriptase fusion protein, wherein the reverse transcriptase fusion protein comprises a reverse transcriptase polypeptide fused to an affinity polypeptide that binds to the RNA recruiting motif, wherein the extended guide nucleic acid binds to a target nucleic acid and the RNA recruiting motif binds to the affinity polypeptide, thereby recruiting the reverse transcriptase fusion protein to the extended guide nucleic acid and contacting the target nucleic acid with the reverse transcriptase. In some embodiments, two or more reverse transcriptase fusion proteins may be recruited to an extended guide nucleic acid, thereby contacting the target nucleic acid with two or more reverse transcriptase fusion proteins.

The terms “transgene” or “transgenic” as used herein refer to at least one nucleic acid sequence that is taken from the genome of one organism or produced synthetically, and which is then introduced into a host cell (e.g., a plant cell) or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation into the genome and thus without inheritance of the respective at least one molecule introduced into the genome of a cell. The term “transgene-free” refers to a condition in which a transgene is not present or found in the genome of a host cell or tissue or organism of interest.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., a polynucleotide encoding a polypeptide (e.g., an engineered protein) of the present invention, a polynucleotide encoding a nuclease, a polynucleotide encoding a reverse transcriptase, a polynucleotide encoding a reverse transcriptase fusion protein, a polynucleotide encoding a peptide tag, a polynucleotide encoding an affinity polypeptide, a polynucleotide encoding a glycosylase, and/or a polynucleotide comprising a guide nucleic acid), wherein the nucleic acid construct is operably associated with at least a control sequence (e.g., a promoter). Thus, some embodiments of the invention provide expression cassettes designed to express, for example, a nucleic acid construct of the invention. When an expression cassette comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). Thus, for example, a polynucleotide encoding a polypeptide of the present invention, a polynucleotide encoding a CRISPR-Cas effector protein and a polynucleotide comprising a guide nucleic acid comprised in an expression cassette may each be operably associated with a single promoter or one or more of the polynucleotide(s) may be operably associated with separate promoters (e.g., two or three promoters) in any combination, which may be the same or different from each other.

In some embodiments, an expression cassette comprising the polynucleotides/nucleic acid constructs of the invention may be optimized for expression in an organism (e.g., an animal, a plant, a bacterium and the like).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to a gene encoding a CRISPR-Cas effector protein, or a gene encoding a polypeptide of the present invention, may be native to a host cell, or may be native to another source (e.g., foreign or heterologous to the promoter, to a gene encoding the CRISPR-Cas effector protein, to a host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

The expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector may comprise a nucleic acid construct comprising one or more nucleotide sequence(s) to be transferred, delivered or introduced into a cell. Vectors for use in transformation of host organisms are well known in the art. Non-limiting examples of general classes of vectors include viral vectors (e.g., Adeno-associated virus (AAV) vectors), plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). Thus, for example, a target nucleic acid may be contacted with a nucleic acid construct of the invention encoding, for example, a nucleic acid binding polypeptide (e.g., a DNA binding domain such as a sequence-specific DNA binding protein (e.g., a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein)), an extended guide nucleic acid, a polynucleotide encoding a polypeptide of the present invention, and optionally a guide nucleic acid, under conditions whereby the nucleic acid binding polypeptide (e.g., a CRISPR-Cas effector protein) is expressed, and the nucleic acid binding polypeptide forms a complex with the extended guide nucleic acid, the complex hybridizes to the target nucleic acid, and optionally the polypeptide of the present invention may be recruited to the nucleic acid binding polypeptide (and thus, to the target nucleic acid) or is/are fused to the nucleic acid binding polypeptide, thereby modifying the target nucleic acid. In some embodiments, the polypeptide of the present invention and the nucleic acid binding polypeptide localize at the target nucleic acid, optionally through covalent and/or non-covalent interactions. Other methods for recruiting a reverse transcriptase may be used that take advantage of other protein-protein interactions, RNA-protein interactions, and/or chemical interactions.

In some embodiments, a target nucleic acid may be contacted with a nucleic acid construct of the invention encoding a polypeptide of the present invention, a CRISPR-Cas effector protein, a guide nucleic acid, under conditions whereby the polypeptide is expressed, or a target nucleic acid may be contacted with polypeptide of the present invention, a CRISPR-Cas effector protein, and a guide nucleic acid. The CRISPR-Cas effector protein can form a complex with the guide nucleic acid, and the complex can hybridize to the target nucleic acid, and/or polypeptide of the present invention is/are recruited to the CRISPR-Cas effector protein (and thus, to the target nucleic acid), and/or polypeptide of the present invention are fused to the CRISPR-Cas effector protein, thereby modifying the target nucleic acid. The polypeptide of the present invention may localize at the target nucleic acid, optionally through covalent and/or non-covalent interactions.

As used herein, “modifying” or “modification” in reference to a target nucleic acid includes editing (e.g., mutating), covalent modification, exchanging/substituting nucleic acids/nucleotide bases, deleting, cleaving, and/or nicking of a target nucleic acid to thereby provide a modified nucleic acid and/or altering transcriptional control of a target nucleic acid to thereby provide a modified nucleic acid. In some embodiments, a modification may include an insertion and/or deletion of any size and/or a single base change (SNP) of any type. In some embodiments, a modification comprises a SNP. In some embodiments, a modification comprises exchanging and/or substituting one or more (e.g., 1, 2, 3, 4, 5, or more) nucleotides. In some embodiments, an insertion or deletion may be about 1 base to about 30,000 bases or more in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000 bases in length or more, or any value or range therein). Thus, in some embodiments, an insertion or deletion may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 bases in length, or any range or value therein; about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 bases to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases or more in length, or any value or range therein; about 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases to about 2500, 3000, 3500, 4000, 4500, 5000, 5500, 6000, 6500, 7000, 7500, 8000, 8500, 9000, 9500, or 10,000 bases or more in length, or any value or range therein; or about 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 bases to about 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, or 5000 bases or more in length, or any value or range therein. In some embodiments, an insertion or deletion may be about 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, or 10,000 bases to about 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, or 30,000 bases or more in length, or any value or range therein.

“Recruit,” “recruiting” or “recruitment” as used herein refer to attracting one or more polypeptide(s) or polynucleotide(s) to another polypeptide or polynucleotide (e.g., to a particular location in a genome) using protein-protein interactions, nucleic acid-protein interactions (e.g., RNA-protein interactions), and/or chemical interactions. Protein-protein interactions can include, but are not limited to, peptide tags (epitopes, multimerized epitopes) and corresponding affinity polypeptides, RNA recruiting motifs and corresponding affinity polypeptides, and/or chemical interactions. Example chemical interactions that may be useful with polypeptides and polynucleotides for the purpose of recruitment can include, but are not limited to, rapamycin-inducible dimerization of FRB-FKBP; Biotin-streptavidin interaction; SNAP tag (Hussain et al. Curr Pharm Des. 19(30):5437-42 (2013)); Halo tag (Los et al. ACS Chem Biol. 3(6):373-82 (2008)); CLIP tag (Gautier et al. Chemistry & Biology 15:128-136 (2008)); DmrA-DmrC heterodimer induced by a compound (Tak et al. Nat Methods 14(12):1163-1166 (2017)); and/or bifunctional ligand approaches e.g., chemically induced dimerization (Voß et al. Curr Opin Chemical Biology 28:194-201 (2015)) (e.g. dihyrofolate reductase (DHFR) (Kopyteck et al. Cell Cehm Biol 7 (5): 313-321 (2000)). In some embodiments, a recruiting method and/or system of the present invention uses a protein-protein interaction and/or nucleic acid-protein interaction (e.g., RNA-protein interactions) to attract a polypeptide or polynucleotide to another polypeptide or polynucleotide (e.g., to a particular location in a genome).

“Introducing,” “introduce,” “introduced” (and grammatical variations thereof) in the context of a polynucleotide of interest or editing system means presenting a nucleotide sequence of interest (e.g., polynucleotide, a nucleic acid construct, and/or a guide nucleic acid) and/or editing system (e.g., a polynucleotide, polypeptide, and/or ribonucleoprotein) to a host organism or cell of said organism (e.g., host cell; e.g., a plant cell) in such a manner that the nucleotide sequence and/or editing system gains access to the interior of a cell. Thus, for example, a nucleic acid construct of the invention encoding a polypeptide of the present invention, a CRISPR-Cas effector protein of the present invention, and/or a guide nucleic acid may be introduced into a cell of an organism, thereby transforming the cell with the polypeptide, CRISPR-Cas effector protein, guide nucleic acid, and reverse transcriptase. In some embodiments, a polypeptide of the present invention and/or a guide nucleic acid may be introduced into a cell of an organism, optionally wherein the polypeptide and guide nucleic acid may be comprised in a complex (e.g., a ribonucleoprotein). In some embodiments, the organism is a eukaryote (e.g., a mammal such as a human).

The term “transformation” as used herein refers to the introduction of a nucleic acid, polypeptide, and/or ribonucleoprotein (e.g., a heterologous nucleic acid, polypeptide, and/or ribonucleoprotein) into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct, a polypeptide, and/or a ribonucleoprotein of the invention.

“Transient transformation” in the context of a polynucleotide, polypeptide, and/or ribonucleoprotein means that a polynucleotide, polypeptide, and/or ribonucleoprotein is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a mammal, plant, etc.). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention may be transiently introduced into a cell with a guide nucleic acid and as such, no DNA maintained in the cell.

A nucleic acid construct, polypeptide, and/or ribonucleoprotein of the invention can be introduced into a cell by any method known to those of skill in the art. In some embodiments, transformation methods include, but are not limited to, transformation via bacterial-mediated nucleic acid delivery (e.g., via Agrobacteria), viral-mediated nucleic acid delivery, silicon carbide and/or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the cell (e.g., a plant cell or an animal cell), including any combination thereof. In some embodiments of the invention, transformation of a cell comprises nuclear transformation. In some embodiments, transformation of a cell comprises plastid transformation (e.g., chloroplast transformation). In some embodiments, a recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

A nucleotide sequence, polypeptide, and/or ribonucleoprotein therefore can be introduced into a host organism or its cell in any number of ways that are well known in the art. The methods of the invention do not depend on a particular method for introducing one or more nucleotide sequence(s), polypeptide(s), and/or ribonucleoprotein(s) into the organism, only that they gain access to the interior of at least one cell of the organism. Where more than one nucleotide sequence, polypeptide, and/or ribonucleoprotein is to be introduced, they can be assembled as part of a single nucleic acid construct, or as separate nucleic acid constructs, and can be located on the same or different nucleic acid constructs. Accordingly, a nucleotide sequence, polypeptide, and/or ribonucleoprotein can be introduced into the cell of interest in a single transformation event, and/or in separate transformation events, or, alternatively, where relevant, a nucleotide sequence can be incorporated into a plant, for example, as part of a breeding protocol. In some embodiments, the cell is a eukaryotic cell (e.g., a plant cell or a mammalian such as a human cell).

In some embodiments, a nucleic acid construct of the invention (e.g., a polynucleotide encoding an CRISPR-Cas effector protein, a polynucleotide encoding a polypeptide of the present invention, and/or a guide nucleic acid and/or expression cassettes and/or vectors comprising the same) may be operably linked to at least one regulatory sequence, optionally, wherein the at least one regulatory sequence may be codon optimized for expression in a plant. In some embodiments, the at least one regulatory sequence may be, for example, a promoter, an operon, a terminator, or an enhancer. In some embodiments, the at least one regulatory sequence may be a promoter. In some embodiments, the regulatory sequence may be an intron. In some embodiments, the at least one regulatory sequence may be, for example, a promoter operably associated with an intron or a promoter region comprising an intron. In some embodiments, the at least one regulatory sequence may be, for example a ubiquitin promoter and its associated intron (e.g., Medicago truncatula and/or Zea mays and their associated introns). In some embodiments, the at least one regulatory sequence may be a terminator nucleotide sequence and/or an enhancer nucleotide sequence.

In some embodiments, a nucleic acid construct of the invention may be operably associated with a promoter region, wherein the promoter region comprises an intron, optionally wherein the promoter region may be a ubiquitin promoter and intron (e.g., a Medicago or a maize ubiquitin promoter and intron, e.g., SEQ ID NO:37 or SEQ ID NO:38). In some embodiments, the nucleic acid construct of the invention that is operably associated with a promoter region comprising an intron may be codon optimized for expression in a plant.

In some embodiments, a nucleic acid construct of the invention may encode one or more (e.g., 1, 2, 3, 4, or more) polypeptide(s) of interest. The one or more polypeptides of interest may be codon optimized for expression in a eukaryote (e.g., a human or a plant). In some embodiments, a polypeptide of the present invention may comprise one or more (e.g., 1, 2, 3, 4, or more) polypeptide(s) of interest.

A polypeptide of interest useful with this invention can include, but is not limited to, a polypeptide or protein domain having deaminase activity, nickase activity, recombinase activity, transposase activity, methylase activity, glycosylase (DNA glycosylase) activity, glycosylase inhibitor activity (e.g., uracil-DNA glycosylase inhibitor (UGI)), a reverse transcriptase, a peptide tag (e.g., a GCN4 peptide tag), demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, a nuclear localization sequence or activity, an affinity polypeptide, a peptide tag, and/or photolyase activity. In some embodiments, the polypeptide of interest is a Fok1 nuclease, or a uracil-DNA glycosylase inhibitor. When encoded in a nucleic acid (polynucleotide, expression cassette, and/or vector) the encoded polypeptide or protein domain may be codon optimized for expression in an organism. In some embodiments, a polypeptide of interest may be linked to a CRISPR-Cas effector protein domain to provide a CRISPR-Cas fusion protein. In some embodiments, a CRISPR-Cas fusion protein that comprises a CRISPR-Cas effector protein domain linked to a peptide tag may also be linked to a polypeptide of interest (e.g., a CRISPR-Cas effector protein domain may be, for example, linked to both a peptide tag (or an affinity polypeptide) and, for example, a polypeptide of interest.

In some embodiments, an editing system of the present invention comprises a CRISPR-Cas effector protein. As used herein, a “CRISPR-Cas effector protein” is a protein or polypeptide that cleaves, cuts, or nicks a nucleic acid; binds a nucleic acid (e.g., a target nucleic acid and/or a guide nucleic acid); and/or that identifies, recognizes, or binds a guide nucleic acid as defined herein. In some embodiments, a CRISPR-Cas effector protein may be an enzyme (e.g., a nuclease, endonuclease, nickase, etc.) and/or may function as an enzyme. In some embodiments, a CRISPR-Cas effector protein refers to a CRISPR-Cas nuclease. In some embodiments, a CRISPR-Cas effector protein comprises nuclease activity and/or nickase activity, comprises a nuclease domain whose nuclease activity and/or nickase activity has been reduced or eliminated, comprises single stranded DNA cleavage activity (ss DNAse activity) or which has ss DNAse activity that has been reduced or eliminated, and/or comprises self-processing RNAse activity or which has self-processing RNAse activity that has been reduced or eliminated. A CRISPR-Cas effector protein may bind to a target nucleic acid. A CRISPR-Cas effector protein may be a Type I, II, III, IV, V, or VI CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein may be from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein. In some embodiments, a CRISPR-Cas effector protein may be Cas12a and optionally may have an amino acid sequence of any one of SEQ ID NOs: 39-65 and/or a nucleotide sequence of any one of SEQ ID NOs: 66-68. In some embodiments, a CRISPR-Cas effector protein may be an active Cas12a and optionally may have an amino acid sequence of SEQ ID NO:49 or 58. In some embodiments, a CRISPR-Cas effector protein may be an inactive (i.e., dead) Cas12a and optionally may have an amino acid sequence of any one of SEQ ID NOs: 39-41. In some embodiments, a CRISPR-Cas effector protein may be Cas12b and optionally may have an amino acid sequence of SEQ ID NO:69. Exemplary CRISPR-Cas effector proteins include, but are not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site and/or nuclease domain (e.g., a RuvC, HNH, e.g., a RuvC site of a Cas12a nuclease domain; e.g., a RuvC site and/or HNH site of a Cas9 nuclease domain). A CRISPR-Cas effector protein having a mutation in its nuclease active site and/or nuclease domain that causes the protein to not have nuclease activity, is commonly referred to as “inactive” or “dead,” e.g., dCas9. In some embodiments, a CRISPR-Cas effector protein having a mutation in its nuclease active site and/or nuclease domain may have impaired activity or reduced activity (e.g., nickase activity) as compared to the same CRISPR-Cas effector protein without the mutation.

A CRISPR Cas9 effector protein or Cas9 useful with this invention may be any known or later identified Cas9 nuclease. In some embodiments, a Cas9 may be a protein from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. In some embodiments, a CRISPR-Cas effector protein may be a Cas9 and optionally may have a nucleotide sequence of any one of SEQ ID NOs: 70-84 and/or an amino acid sequence of any one of SEQ ID NOs: 85-94.

In some embodiments, the CRISPR-Cas effector protein may be a Cas9 derived from Streptococcus pyogenes and/or may recognize the PAM sequence motif NGG, NAG, NGA (Mali et al, Science 2013; 339 (6121): 823-826). In some embodiments, the CRISPR-Cas effector protein may be a Cas9 derived from Streptococcus thermophiles and/or may recognize the PAM sequence motif NGGNG and/or NNAGAAW (W=A or T) (See, e.g., Horvath et al, Science, 2010; 327 (5962): 167-170, and Deveau et al, J Bacteriol 2008; 190 (4): 1390-1400). In some embodiments, the CRISPR-Cas effector protein may be a Cas9 derived from Streptococcus mutans and/or may recognize the PAM sequence motif NGG and/or NAAR (R=A or G) (See, e.g., Deveau et al, J BACTERIOL 2008; 190 (4): 1390-1400). In some embodiments, the CRISPR-Cas effector protein may be a Cas9 derived from Streptococcus aureus and/or may recognize the PAM sequence motif NNGRR (R=A or G). In some embodiments, the CRISPR-Cas effector protein may be a Cas9 derived from S. aureus and/or may recognize the PAM sequence motif N GRRT (R=A or G). In some embodiments, the CRISPR-Cas effector protein may be a Cas9 derived from S. aureus and/or may recognize the PAM sequence motif N GRRV (R=A or G). In some embodiments, the CRISPR-Cas effector protein may be a Cas9 that is derived from Neisseria meningitidis and/or may recognize the PAM sequence motif N GATT or N GCTT (R=A or G, V=A, G or C) (See, e.g., Hou et ah, PNAS 2013, 1-6). In the aforementioned embodiments in this paragraph, N in the PAM sequence motif can be any nucleotide residue, e.g., any of A, G, C or T. In some embodiments, the CRISPR-Cas effector protein may be a Cas13a derived from Leptotrichia shahii and/or may recognize a protospacer flanking sequence (PFS) (or RNA PAM (rPAM)) sequence motif of a single 3′ A, U, or C, which may be located within the target nucleic acid.

A Type V CRISPR-Cas effector protein useful with embodiments of the invention may be any Type V CRISPR-Cas nuclease. Exemplary Type V CRISPR-Cas effector proteins include, but are not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas effector protein may be a Cas12a. In some embodiments, a Type V CRISPR-Cas effector protein may be a nickase, optionally, a Cas12a nickase. In some embodiments, a Type V CRISPR-Cas effector protein may be a Cas12b (e.g., SEQ ID NO:69).

In some embodiments, the CRISPR-Cas effector protein may be a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein useful with this invention may be any known or later identified Cas12a (previously known as Cpf1) (see, e.g., U.S. Pat. No. 9,790,490, which is incorporated by reference for its disclosures of Cpf1 (Cas12a) sequences). The term “Cas12a” refers to an RNA-guided protein that can have nuclease activity, the protein comprising a guide nucleic acid binding domain and an active, inactive, or partially active DNA cleavage domain, thereby the RNA-guided nuclease activity of the Cas12a may be active, inactive or partially active, respectively. In some embodiments, a Cas12a useful with the invention may comprise a mutation in the nuclease active site (e.g., a RuvC site of the Cas12a domain). A Cas12a having a mutation in its nuclease domain and/or nuclease active site, and therefore, no longer comprising nuclease activity, is commonly referred to as deadCas 12a (e.g., dCas12a). In some embodiments, a Cas12a having a mutation in its nuclease domain and/or nuclease active site may have impaired activity, e.g., may have reduced nickase activity. In some embodiments, a Cas12a may have an amino acid sequence of any one of SEQ ID NOs: 39-65.

In some embodiments, a CRISPR-Cas effector protein may be optimized for expression in an organism, for example, in an animal (e.g., a mammal such as a human), a plant, a fungus, an archaeon, or a bacterium. In some embodiments, a CRISPR-Cas effector protein (e.g., Cas12a polypeptide/domain or a Cas9 polypeptide/domain) may be optimized for expression in a plant.

In some embodiments, a CRISPR-Cas effector protein (e.g., an engineered CRISPR-Cas effector protein) is a target strand nickase and/or a non-target strand nickase. In some embodiments, a CRISPR-Cas effector protein (e.g., an engineered CRISPR-Cas effector protein) is a non-target strand nickase. In some embodiments, a CRISPR-Cas effector protein is an engineered protein that has an amino acid sequence having at least 70%, 75%, 80%, 85%, 90%, 95%, 96%, 97%, 98%, 99%, or 100% sequence identity to one or more of SEQ ID NOs: 410-501.

A polypeptide of the present invention may be used in combination with a guide nucleic acid (e.g., guide RNA (gRNA), CRISPR array, CRISPR RNA, crRNA, or extended guide nucleic acid) that is designed to function with a CRISPR-Cas effector protein to modify a target nucleic acid. A guide nucleic acid useful with this invention may comprise at least one spacer sequence and at least one repeat sequence. The guide nucleic acid may be capable of forming a complex with a CRISPR-Cas effector protein (e.g., with a nuclease domain of the protein) and the spacer sequence is capable of hybridizing to a target nucleic acid, thereby guiding the complex to the target nucleic acid, wherein the target nucleic acid may be modified (e.g., cleaved or edited) and/or modulated (e.g., modulating transcription) by a polypeptide of the present invention, optionally present in and/or recruited to the complex).

In some embodiments, a CRISPR-Cas effector protein comprising a Cas9 domain (or a nucleic acid construct encoding the same) may be used in combination with a Cas9 guide nucleic acid to modify a target nucleic acid, and may be in or may form a complex.

Likewise, a CRISPR-Cas effector protein may comprise a Cas12a domain (or other selected CRISPR-Cas nuclease, e.g., C2c1, C2c3, Cas12b, Cas12c, Cas12d, Cas12f, Cas12i, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5), which may be used in combination with a Cas 12a guide nucleic acid (or the guide nucleic acid for the other selected CRISPR-Cas nuclease) to modify a target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target nucleic acid (e.g., a target DNA and/or a protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas12f, Cas12i, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2cl locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild-type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild-type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be fully complementary to a target nucleic acid, while the 3′ region of the spacer may be substantially complementary to the target nucleic acid (such as for a spacer in a Type V CRISPR-Cas system), or the 3′ region of a spacer sequence of a guide nucleic acid may be fully complementary to a target nucleic acid, while the 5′ region of the spacer may be substantially complementary to the target nucleic acid (such as for a spacer in a Type II CRISPR-Cas system), and therefore, the overall complementarity of the spacer sequence to the target nucleic acid may be less than 100%. Thus, for example, in a guide nucleic acid for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target nucleic acid, while the remaining nucleotides in the 3′ region of the spacer sequence are substantially complementary (e.g., at least about 70% complementary) to the target nucleic acid. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target nucleic acid, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleic acid.

As a further example, in a guide nucleic acid for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target nucleic acid, while the remaining nucleotides in the 5′ region of the spacer sequence are substantially complementary (e.g., at least about 70% complementary) to the target nucleic acid. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target nucleic acid, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target nucleic acid. A recruiting guide RNA further comprises one or more recruiting motifs as described herein, which may be linked to the 5′ end of the guide or the 3′ end or it may be inserted into the recruiting guide nucleic acid (e.g., within the hairpin loop).

In some embodiments, a seed region of a spacer may be about 8 to about 10 nucleotides in length, about 5 to about 6 nucleotides in length, or about 6 nucleotides in length.

In some embodiments, a guide nucleic acid further comprises a reverse transcriptase template and may be referred to as an extended guide nucleic acid.

A guide nucleic acid and/or an extended guide nucleic acid may comprise one or more recruiting motifs as described herein, which may be linked to the 5′ end and/or the 3′ end of the guide nucleic acid and/or it may be inserted into the guide nucleic acid (e.g., within a hairpin loop of the guide nucleic acid).

A “target nucleic acid”, “target DNA,” “target nucleotide sequence,” “target region,” and “target region in the genome” are used interchangeably herein and refer to a region of an organism's (e.g., a plant's) genome that comprises a sequence that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid as defined herein. A target nucleic acid is targeted by an editing system (or a component thereof) as described herein. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome or mammalian (e.g., human) genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” or “protospacer” as used herein refer to a sequence that is fully or substantially complementary to (and can hybridize to) a spacer sequence of a guide nucleic acid. In some embodiments, the protospacer is all or a portion of a target nucleic acid as defined herein that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
    5′-NNNNNNNNNNNNNNNNNNN-3′ RNA Spacer
          |||||||||||||||||||
3′AAANNNNNNNNNNNNNNNNNNN-5′ Target strand
    ||||
5′TTTNNNNNNNNNNNNNNNNNNN-3′ Non-target strand
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encodes a polypeptide of the present invention and/or a CRISPR-Cas effector protein, and each may be comprised on the same or on a separate expression cassette or vector from that comprising the one or more guide nucleic acids. When the nucleic acid construct encoding a polypeptide of the present invention or the components of an editing system is/are comprised on separate expression cassette(s) or vector(s) from that comprising the guide nucleic acid, a target nucleic acid may be contacted with (e.g., provided with) the expression cassette(s) or vector(s) encoding the polypeptide of the present invention or components of an editing system in any order from one another and the guide nucleic acid, e.g., prior to, concurrently with, or after the expression cassette comprising the guide nucleic acid is provided (e.g., contacted with the target nucleic acid).

Methods of recruiting one or more components of an editing system to each other and/or to a target nucleic acid are known in the art and may include the use of a peptide tag or an affinity polypeptide that interacts with the peptide tag. In some embodiments, a guide nucleic acid may be linked to an RNA recruiting motif and a polypeptide of the present invention may be linked to an affinity polypeptide capable of interacting with the RNA recruiting motif, thereby recruiting the polypeptide of the invention to the target nucleic acid. Alternatively, chemical interactions may be used to recruit a polypeptide (e.g., a polypeptide of the invention) to a target nucleic acid.

A “recruiting motif” as used herein refers to one half of a binding pair that may be used to recruit a compound to which the recruiting motif is bound to another compound that includes the other half of the binding pair (i.e., a “corresponding motif”). The recruiting motif and corresponding motif may bind noncovalently. In some embodiments, a recruiting motif is an RNA recruiting motif (e.g., an RNA recruiting motif that is capable of binding and/or configured to bind to an affinity polypeptide), an affinity polypeptide (e.g., an affinity polypeptide that is capable of binding and/or configured to bind an RNA recruiting motif and/or a peptide tag), or a peptide tag (e.g., a peptide tag that is capable of binding and/or configured to bind an affinity polypeptide). For example, when a recruiting motif is an RNA recruiting motif, the corresponding motif for the RNA recruiting motif may be an affinity polypeptide that binds the RNA recruiting motif. A further example is that when a recruiting motif is a peptide tag, the corresponding motif for the peptide tag may be an affinity polypeptide that binds the peptide tag. Thus, a compound comprising a recruiting motif (e.g., an affinity polypeptide) may be recruited to another compound (e.g., a guide nucleic acid) comprising a corresponding motif for the recruiting motif (e.g., an RNA recruiting motif).

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a polypeptide of the present invention) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., a polypeptide of the present invention). In some embodiments, two or more polypeptides may be recruited to a guide nucleic acid, thereby contacting the target nucleic acid with two or more polypeptides (e.g., one or more polypeptide(s) of the present invention).

In some embodiments of the invention, a guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF). Exemplary RNA recruiting motifs and corresponding affinity polypeptides that may be useful with this invention can include, but are not limited to, SEQ ID NOs: 95-105.

In some embodiments, the components for recruiting polypeptides and nucleic acids may include those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., chemically induced dimerization).

As described herein, a “peptide tag” may be employed to recruit one or more polypeptides. A peptide tag may be any polypeptide that is capable of being bound by a corresponding motif such as an affinity polypeptide. A peptide tag may also be referred to as an “epitope” and when provided in multiple copies, a “multimerized epitope.” Example peptide tags can include, but are not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO2018/136783 and U.S. Patent Application Publication No. 2017/0219596, which are incorporated by reference for their disclosures of peptide tags. Peptide tags that may be useful with this invention can include, but are not limited to, SEQ ID NOs: 106 and 107. An affinity polypeptide useful with peptide tags includes, but is not limited to, SEQ ID NO:108.

A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20 or more amino acids, optionally about 3 to about 10, about 4 to about 10, about 5 to about 10, about 5 to about 15, or about 5 to about 20 amino acids, and the like, and any value or range therein. Thus, in some embodiments, a CRISPR-Cas effector protein and/or polypeptide of the invention may be fused to one peptide tag or to two or more peptide tags, optionally wherein the two or more peptide tags are fused to one another via one or more amino acid residues. In some embodiments, a peptide tag useful with the invention may be a single copy of a GCN4 peptide tag or epitope or may be a multimerized GCN4 epitope comprising about 2 to about 25 or more copies of the peptide tag (e.g., about 2, 3, 4, 5, 6, 7, 8, 9, 10, 11, 12, 13, 14, 15, 16, 17, 18, 19, 20, 21, 22, 23, 24, 25 or more copies of a GCN4 epitope or any range therein).

In some embodiments, a peptide tag may be fused to a CRISPR-Cas polypeptide or domain. In some embodiments, a peptide tag may be fused or linked to the C-terminus of a CRISPR-Cas effector protein to form a CRISPR-Cas fusion protein. In some embodiments, a peptide tag may be fused or linked to the N-terminus of a CRISPR-Cas effector protein to form a CRISPR-Cas fusion protein. In some embodiments, a peptide tag may be fused within a CRISPR-Cas effector protein (e.g., a peptide tag may be in a loop region of a CRISPR-Cas effector protein). In some embodiments, peptide tag may be fused to or a polypeptide of the present invention.

An “affinity polypeptide” (e.g., “recruiting polypeptide”) refers to any polypeptide that is capable of binding to its corresponding peptide tag, peptide tag, or RNA recruiting motif. An affinity polypeptide for a peptide tag may be, for example, an antibody and/or a single chain antibody that specifically binds the peptide tag, respectively. In some embodiments, an antibody for a peptide tag may be, but is not limited to, an scFv antibody. In some embodiments, an affinity polypeptide may be fused or linked to the N-terminus of a polypeptide of the present invention. In some embodiments, the affinity polypeptide is stable under the reducing conditions of a cell or cellular extract.

A fusion protein of the present invention may comprise a nuclear localization signal (NLS). In some embodiments, an NLS has at least 70%, 75%, 80%, 85%, 90%, 95%, 96%, 97%, 98%, 99%, or 100% sequence identity to one or more of SEQ ID NOs: 109-114. In some embodiments, an NLS of the present invention is encoded by a polynucleotide having at least 70%, 75%, 80%, 85%, 90%, 95%, 96%, 97%, 98%, 99%, or 100% sequence identity to one or more of any one of SEQ ID NOs: 115-119. In some embodiments, a fusion protein of the present invention comprises a NLS that is fused (directly or indirectly) to the N- or C-terminus of the fusion protein. In some embodiments, a NLS is present at the N-terminus and/or C-terminus of a fusion protein of the present invention.

The nucleic acid constructs of the invention and/or guide nucleic acids may be comprised in one or more expression cassettes as described herein. In some embodiments, a nucleic acid construct of the invention may be comprised in the same or in a separate expression cassette or vector from that comprising a guide nucleic acid and/or an extended guide nucleic acid.

In some embodiments, a nucleic acid construct, expression cassette, or vector of the invention that is optimized for expression in an organism (e.g., a human or plant) may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to a nucleic acid construct, expression cassette or vector comprising the same polynucleotide(s) but which have not been codon optimized for expression in the organism.

When used in combination with a guide nucleic acid, a nucleic acid construct of the invention (and expression cassette and/or vector comprising the same) may be used to modify a target nucleic acid and/or its expression. A target nucleic acid may be contacted with a nucleic acid construct of the invention and/or expression cassettes and/or vectors comprising the same prior to, concurrently with or after contacting the target nucleic acid with the guide nucleic acid/recruiting guide nucleic acid (and/or expression cassettes and vectors comprising the same.

According to embodiments of the present invention, provided herein are polypeptides (e.g., engineered proteins). An “engineered protein” as used herein refers to a polypeptide or protein that is not found naturally in nature. An engineered protein may be referred to as a mutant protein. In some embodiments, a mutant protein comprises a non-natural mutation.

In some embodiments, a polypeptide of the present invention comprises an amino acid sequence having at least 70%, 75%, 80%, 85%, 90%, 95%, 96%, 97%, 98%, 99%, or more sequence identity to SEQ ID NO:258 when the amino acid sequence is optimally aligned thereto, wherein the amino acid sequence, when optimally aligned to SEQ ID NO:258, has: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; a D at an amino acid residue corresponding to amino acid residue 148 of SEQ ID NO:258; an A, N, D, R, C, E, Q, G, I, leucine (L), K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; an A, N, D, R, C, Q, G, H, I, L, K, M, F, P, S, T, W, Y, or V at an amino acid residue corresponding to amino acid residue 171 of SEQ ID NO:258; a D at an amino acid residue corresponding to amino acid residue 181 of SEQ ID NO:258; an R at an amino acid residue corresponding to amino acid residue 221 of SEQ ID NO:258; a G at an amino acid residue corresponding to amino acid residue 225 of SEQ ID NO:258; a V at an amino acid residue corresponding to amino acid residue 230 of SEQ ID NO:258; an F at an amino acid residue corresponding to amino acid residue 234 of SEQ ID NO:258; an R or S at an amino acid residue corresponding to amino acid residue 236 of SEQ ID NO:258; an H at an amino acid residue corresponding to amino acid residue 237 of SEQ ID NO:258; a G at an amino acid residue corresponding to amino acid residue 241 of SEQ ID NO:258; an H at an amino acid residue corresponding to amino acid residue 252 of SEQ ID NO:258; a V at an amino acid residue corresponding to amino acid residue 256 of SEQ ID NO:258; a D at an amino acid residue corresponding to amino acid residue 275 of SEQ ID NO:258; an H at an amino acid residue corresponding to amino acid residue 287 of SEQ ID NO:258; an R at an amino acid residue corresponding to amino acid residue 289 of SEQ ID NO:258; an F at an amino acid residue corresponding to amino acid residue 325 of SEQ ID NO:258; an R at an amino acid residue corresponding to amino acid residue 327 of SEQ ID NO:258; an A at an amino acid residue corresponding to amino acid residue 332 of SEQ ID NO:258; and/or a Y at an amino acid residue corresponding to amino acid residue 335 of SEQ ID NO:258. In some embodiments, the polypeptide generates DNA (e.g., cDNA) from RNA. In some embodiments, the polypeptide comprises an amino acid sequence having at least 95%, 96%, 97%, 98%, 99%, 99.5% or more sequence identity to SEQ ID NO:258 when the amino acid sequence is optimally aligned thereto. The polypeptide may also have an N at an amino acid residue corresponding to amino acid residue 200 of SEQ ID NO: 258, a K at an amino acid residue corresponding to amino acid residue 306 of SEQ ID NO: 258, an F at an amino acid residue corresponding to amino acid residue 313 of SEQ ID NO: 258, a P at an amino acid residue corresponding to amino acid residue 330 of SEQ ID NO: 258, and/or a W at an amino acid residue corresponding to amino acid residue 603 of SEQ ID NO: 258. In some embodiments, the polypeptide has an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide has an A, N, D, R, C, E, Q, G, H, I, L, K, M, F, P, S, T, W, or Y at an amino acid residue corresponding to amino acid residue 223 of SEQ ID NO:258.

In some embodiments, the polypeptide comprises an amino acid sequence having at least 70% sequence identity to SEQ ID NO:258 when the amino acid sequence is optimally aligned thereto, wherein the amino acid sequence, when optimally aligned to SEQ ID NO:258, has one or more (e.g., 1, 2, 3, 4, 5, or more) mutation(s) selected from: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; a D at an amino acid residue corresponding to amino acid residue 148 of SEQ ID NO:258; an A, N, D, R, C, E, Q, G, I, leucine (L), K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; an A, N, D, R, C, Q, G, H, I, L, K, M, F, P, S, T, W, Y, or V at an amino acid residue corresponding to amino acid residue 171 of SEQ ID NO: 258; a D at an amino acid residue corresponding to amino acid residue 181 of SEQ ID NO: 258; an R at an amino acid residue corresponding to amino acid residue 221 of SEQ ID NO: 258; a G at an amino acid residue corresponding to amino acid residue 225 of SEQ ID NO: 258; a V at an amino acid residue corresponding to amino acid residue 230 of SEQ ID NO: 258; an F at an amino acid residue corresponding to amino acid residue 234 of SEQ ID NO: 258; an R or S at an amino acid residue corresponding to amino acid residue 236 of SEQ ID NO: 258; an H at an amino acid residue corresponding to amino acid residue 237 of SEQ ID NO: 258; a G at an amino acid residue corresponding to amino acid residue 241 of SEQ ID NO: 258; an H at an amino acid residue corresponding to amino acid residue 252 of SEQ ID NO: 258; a V at an amino acid residue corresponding to amino acid residue 256 of SEQ ID NO: 258; a D at an amino acid residue corresponding to amino acid residue 275 of SEQ ID NO: 258; an H at an amino acid residue corresponding to amino acid residue 287 of SEQ ID NO: 258; an R at an amino acid residue corresponding to amino acid residue 289 of SEQ ID NO: 258; an F at an amino acid residue corresponding to amino acid residue 325 of SEQ ID NO: 258; an R at an amino acid residue corresponding to amino acid residue 327 of SEQ ID NO: 258; an A at an amino acid residue corresponding to amino acid residue 332 of SEQ ID NO: 258; and a Y at an amino acid residue corresponding to amino acid residue 335 of SEQ ID NO: 258, and wherein the polypeptide comprises an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258.

In embodiments, the polypeptide comprises an amino acid sequence having at least 70% sequence identity to SEQ ID NO:258 when the amino acid sequence is optimally aligned thereto, wherein the amino acid sequence, when optimally aligned to SEQ ID NO:258, has only one mutation selected from: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; a D at an amino acid residue corresponding to amino acid residue 148 of SEQ ID NO:258; an A, N, D, R, C, E, Q, G, I, leucine (L), K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; an A, N, D, R, C, Q, G, H, I, L, K, M, F, P, S, T, W, Y, or V at an amino acid residue corresponding to amino acid residue 171 of SEQ ID NO:258; a D at an amino acid residue corresponding to amino acid residue 181 of SEQ ID NO:258; an R at an amino acid residue corresponding to amino acid residue 221 of SEQ ID NO:258; a G at an amino acid residue corresponding to amino acid residue 225 of SEQ ID NO:258; a V at an amino acid residue corresponding to amino acid residue 230 of SEQ ID NO:258; an F at an amino acid residue corresponding to amino acid residue 234 of SEQ ID NO:258; an R or S at an amino acid residue corresponding to amino acid residue 236 of SEQ ID NO:258; an H at an amino acid residue corresponding to amino acid residue 237 of SEQ ID NO:258; a G at an amino acid residue corresponding to amino acid residue 241 of SEQ ID NO:258; an H at an amino acid residue corresponding to amino acid residue 252 of SEQ ID NO:258; a V at an amino acid residue corresponding to amino acid residue 256 of SEQ ID NO:258; a D at an amino acid residue corresponding to amino acid residue 275 of SEQ ID NO:258; an H at an amino acid residue corresponding to amino acid residue 287 of SEQ ID NO:258; an R at an amino acid residue corresponding to amino acid residue 289 of SEQ ID NO:258; an F at an amino acid residue corresponding to amino acid residue 325 of SEQ ID NO:258; an R at an amino acid residue corresponding to amino acid residue 327 of SEQ ID NO:258; an A at an amino acid residue corresponding to amino acid residue 332 of SEQ ID NO:258; and a Y at an amino acid residue corresponding to amino acid residue 335 of SEQ ID NO:258 (i.e., the polypeptide has one mutation from the list and the other positions in the list are not mutated), and wherein the polypeptide comprises an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258.

In embodiments, the polypeptide comprises an amino acid sequence having at least 70% sequence identity to SEQ ID NO:258 when the amino acid sequence is optimally aligned thereto, wherein the amino acid sequence, when optimally aligned to SEQ ID NO:258, has two mutations that are each selected from: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; a D at an amino acid residue corresponding to amino acid residue 148 of SEQ ID NO:258; an A, N, D, R, C, E, Q, G, I, leucine (L), K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; an A, N, D, R, C, Q, G, H, I, L, K, M, F, P, S, T, W, Y, or V at an amino acid residue corresponding to amino acid residue 171 of SEQ ID NO:258; a D at an amino acid residue corresponding to amino acid residue 181 of SEQ ID NO:258; an R at an amino acid residue corresponding to amino acid residue 221 of SEQ ID NO:258; a G at an amino acid residue corresponding to amino acid residue 225 of SEQ ID NO:258; a V at an amino acid residue corresponding to amino acid residue 230 of SEQ ID NO:258; an F at an amino acid residue corresponding to amino acid residue 234 of SEQ ID NO:258; an R or S at an amino acid residue corresponding to amino acid residue 236 of SEQ ID NO:258; an H at an amino acid residue corresponding to amino acid residue 237 of SEQ ID NO:258; a G at an amino acid residue corresponding to amino acid residue 241 of SEQ ID NO:258; an H at an amino acid residue corresponding to amino acid residue 252 of SEQ ID NO:258; a V at an amino acid residue corresponding to amino acid residue 256 of SEQ ID NO:258; a D at an amino acid residue corresponding to amino acid residue 275 of SEQ ID NO:258; an H at an amino acid residue corresponding to amino acid residue 287 of SEQ ID NO:258; an R at an amino acid residue corresponding to amino acid residue 289 of SEQ ID NO:258; an F at an amino acid residue corresponding to amino acid residue 325 of SEQ ID NO:258; an R at an amino acid residue corresponding to amino acid residue 327 of SEQ ID NO:258; an A at an amino acid residue corresponding to amino acid residue 332 of SEQ ID NO:258; and a Y at an amino acid residue corresponding to amino acid residue 335 of SEQ ID NO:258 (i.e., the polypeptide has two mutations from the list and the other positions in the list are not mutated), and wherein the polypeptide comprises an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258.

In embodiments, the polypeptide comprises an amino acid sequence having at least 70% sequence identity to SEQ ID NO:258 when the amino acid sequence is optimally aligned thereto, wherein the amino acid sequence, when optimally aligned to SEQ ID NO:258, has three mutations that are each selected from: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; a D at an amino acid residue corresponding to amino acid residue 148 of SEQ ID NO:258; an A, N, D, R, C, E, Q, G, I, leucine (L), K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; an A, N, D, R, C, Q, G, H, I, L, K, M, F, P, S, T, W, Y, or V at an amino acid residue corresponding to amino acid residue 171 of SEQ ID NO:258; a D at an amino acid residue corresponding to amino acid residue 181 of SEQ ID NO:258; an R at an amino acid residue corresponding to amino acid residue 221 of SEQ ID NO:258; a G at an amino acid residue corresponding to amino acid residue 225 of SEQ ID NO:258; a V at an amino acid residue corresponding to amino acid residue 230 of SEQ ID NO:258; an F at an amino acid residue corresponding to amino acid residue 234 of SEQ ID NO:258; an R or S at an amino acid residue corresponding to amino acid residue 236 of SEQ ID NO:258; an H at an amino acid residue corresponding to amino acid residue 237 of SEQ ID NO:258; a G at an amino acid residue corresponding to amino acid residue 241 of SEQ ID NO:258; an H at an amino acid residue corresponding to amino acid residue 252 of SEQ ID NO:258; a V at an amino acid residue corresponding to amino acid residue 256 of SEQ ID NO:258; a D at an amino acid residue corresponding to amino acid residue 275 of SEQ ID NO:258; an H at an amino acid residue corresponding to amino acid residue 287 of SEQ ID NO:258; an R at an amino acid residue corresponding to amino acid residue 289 of SEQ ID NO:258; an F at an amino acid residue corresponding to amino acid residue 325 of SEQ ID NO:258; an R at an amino acid residue corresponding to amino acid residue 327 of SEQ ID NO:258; an A at an amino acid residue corresponding to amino acid residue 332 of SEQ ID NO:258; and a Y at an amino acid residue corresponding to amino acid residue 335 of SEQ ID NO:258 (i.e., the polypeptide has three mutations from the list and the other positions in the list are not mutated), and wherein the polypeptide comprises an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258.

In embodiments, the polypeptide comprises an amino acid sequence having at least 70% sequence identity to SEQ ID NO:258 when the amino acid sequence is optimally aligned thereto, wherein the amino acid sequence, when optimally aligned to SEQ ID NO:258, has four mutations that are each selected from: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; a D at an amino acid residue corresponding to amino acid residue 148 of SEQ ID NO:258; an A, N, D, R, C, E, Q, G, I, leucine (L), K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; an A, N, D, R, C, Q, G, H, I, L, K, M, F, P, S, T, W, Y, or V at an amino acid residue corresponding to amino acid residue 171 of SEQ ID NO:258; a D at an amino acid residue corresponding to amino acid residue 181 of SEQ ID NO:258; an R at an amino acid residue corresponding to amino acid residue 221 of SEQ ID NO:258; a G at an amino acid residue corresponding to amino acid residue 225 of SEQ ID NO:258; a V at an amino acid residue corresponding to amino acid residue 230 of SEQ ID NO:258; an F at an amino acid residue corresponding to amino acid residue 234 of SEQ ID NO:258; an R or S at an amino acid residue corresponding to amino acid residue 236 of SEQ ID NO:258; an H at an amino acid residue corresponding to amino acid residue 237 of SEQ ID NO:258; a G at an amino acid residue corresponding to amino acid residue 241 of SEQ ID NO:258; an H at an amino acid residue corresponding to amino acid residue 252 of SEQ ID NO:258; a V at an amino acid residue corresponding to amino acid residue 256 of SEQ ID NO:258; a D at an amino acid residue corresponding to amino acid residue 275 of SEQ ID NO:258; an H at an amino acid residue corresponding to amino acid residue 287 of SEQ ID NO:258; an R at an amino acid residue corresponding to amino acid residue 289 of SEQ ID NO:258; an F at an amino acid residue corresponding to amino acid residue 325 of SEQ ID NO:258; an R at an amino acid residue corresponding to amino acid residue 327 of SEQ ID NO:258; an A at an amino acid residue corresponding to amino acid residue 332 of SEQ ID NO:258; and a Y at an amino acid residue corresponding to amino acid residue 335 of SEQ ID NO:258 (i.e., the polypeptide has four mutations from the list and the other positions in the list are not mutated), and wherein the polypeptide comprises an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258.

In some embodiments, a polypeptide of the present invention comprises an amino acid sequence having at least 70%, 75%, 80%, 85%, 90%, 95%, 96%, 97%, 98%, 99%, or more sequence identity to SEQ ID NO:258 when the amino acid sequence is optimally aligned thereto, and wherein the amino acid sequence, when optimally aligned to SEQ ID NO:258, has: a D at an amino acid residue corresponding to amino acid residue 148 of SEQ ID NO:258; a D at an amino acid residue corresponding to amino acid residue 181 of SEQ ID NO:258; an R at an amino acid residue corresponding to amino acid residue 221 of SEQ ID NO:258; an F at an amino acid residue corresponding to amino acid residue 234 of SEQ ID NO:258; an R or S at an amino acid residue corresponding to amino acid residue 236 of SEQ ID NO:258; a G at an amino acid residue corresponding to amino acid residue 241 of SEQ ID NO:258; an R at an amino acid residue corresponding to amino acid residue 289 of SEQ ID NO:258; and/or an R at an amino acid residue corresponding to amino acid residue 327 of SEQ ID NO:258. The polypeptide may also have an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and/or a W at an amino acid residue corresponding to amino acid residue 603 of SEQ ID NO:258. In some embodiments, the polypeptide has an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide has an A, N, D, R, C, E, Q, G, H, I, L, K, M, F, P, S, T, W, or Y at an amino acid residue corresponding to amino acid residue 223 of SEQ ID NO:258.

In some embodiments, a polypeptide of the present invention comprises an amino acid sequence having at least 70%, 75%, 80%, 85%, 90%, 95%, 96%, 97%, 98%, 99%, or more sequence identity to SEQ ID NO:258 when the amino acid sequence is optimally aligned thereto, and wherein the amino acid sequence, when optimally aligned to SEQ ID NO:258, has: a G at an amino acid residue corresponding to amino acid residue 225 of SEQ ID NO:258 and an H at an amino acid residue corresponding to amino acid residue 237 of SEQ ID NO: 258; a V at an amino acid residue corresponding to amino acid residue 230 of SEQ ID NO: 258, a V at an amino acid residue corresponding to amino acid residue 256 of SEQ ID NO: 258, and a D at an amino acid residue corresponding to amino acid residue 275 of SEQ ID NO: 258; and/or an H at an amino acid residue corresponding to amino acid residue 252 of SEQ ID NO:258, an F at an amino acid residue corresponding to amino acid residue 325 of SEQ ID NO:258, an A at an amino acid residue corresponding to amino acid residue 332 of SEQ ID NO:258, and a Y at an amino acid residue corresponding to amino acid residue 335 of SEQ ID NO:258. The polypeptide may also have an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and/or a W at an amino acid residue corresponding to amino acid residue 603 of SEQ ID NO:258. In some embodiments, the polypeptide has an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide has an A, N, D, R, C, E, Q, G, H, I, L, K, M, F, P, S, T, W, or Y at an amino acid residue corresponding to amino acid residue 223 of SEQ ID NO:258. In some embodiments, the polypeptide has a G at an amino acid residue corresponding to amino acid residue 225 of SEQ ID NO:258 and an H at an amino acid residue corresponding to amino acid residue 237 of SEQ ID NO:258. In some embodiments, the polypeptide has a V at an amino acid residue corresponding to amino acid residue 230 of SEQ ID NO:258, a V at an amino acid residue corresponding to amino acid residue 256 of SEQ ID NO:258, and a D at an amino acid residue corresponding to amino acid residue 275 of SEQ ID NO:258. In some embodiments, the polypeptide has an H at an amino acid residue corresponding to amino acid residue 252 of SEQ ID NO:258, an F at an amino acid residue corresponding to amino acid residue 325 of SEQ ID NO:258, an A at an amino acid residue corresponding to amino acid residue 332 of SEQ ID NO:258, and a Y at an amino acid residue corresponding to amino acid residue 335 of SEQ ID NO:258.

In some embodiments, a polypeptide of the present invention comprises an amino acid sequence having at least 70%, 75%, 80%, 85%, 90%, 95%, 96%, 97%, 98%, 99%, or more sequence identity to SEQ ID NO:258 when the amino acid sequence is optimally aligned thereto, and wherein the amino acid sequence, when optimally aligned to SEQ ID NO:258, has a C, M, or V at an amino acid residue corresponding to amino acid residue 168 of SEQ ID NO: 258. The polypeptide may also have an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and/or a W at an amino acid residue corresponding to amino acid residue 603 of SEQ ID NO:258. In some embodiments, the polypeptide has an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide has an A, N, D, R, C, E, Q, G, H, I, L, K, M, F, P, S, T, W, or Y at an amino acid residue corresponding to amino acid residue 223 of SEQ ID NO:258.

In some embodiments, a polypeptide of the present invention comprises an amino acid sequence having at least 70%, 75%, 80%, 85%, 90%, 95%, 96%, 97%, 98%, 99%, or more sequence identity to SEQ ID NO:258 when the amino acid sequence is optimally aligned thereto, and wherein the amino acid sequence, when optimally aligned to SEQ ID NO:258, has: an A, N, D, R, C, E, Q, G, H, I, K, M, F, P, S, T, W, Y, or V at an amino acid residue corresponding to amino acid residue 139 of SEQ ID NO:258; an A, N, D, R, C, E, Q, G, I, L, K, M, F, P, S, T, W, Y, or V at an amino acid residue corresponding to amino acid residue 161 of SEQ ID NO:258; a C, M, or V at an amino acid residue corresponding to amino acid residue 168 of SEQ ID NO:258; and/or an A, N, D, R, C, Q, G, H, I, L, K, M, F, P, S, T, W, Y, or V at an amino acid residue corresponding to amino acid residue 171 of SEQ ID NO:258. The polypeptide may also have an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and/or a W at an amino acid residue corresponding to amino acid residue 603 of SEQ ID NO:258. In some embodiments, the polypeptide has an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide has an A, N, D, R, C, E, Q, G, H, I, L, K, M, F, P, S, T, W, or Y at an amino acid residue corresponding to amino acid residue 223 of SEQ ID NO:258. In some embodiments, the polypeptide has an A, N, D, R, C, E, Q, G, H, I, K, M, F, P, S, T, W, Y, or V at an amino acid residue corresponding to amino acid residue 139 of SEQ ID NO:258. In some embodiments, the polypeptide has an A, N, D, R, C, E, Q, G, I, L, K, M, F, P, S, T, W, Y, or V at an amino acid residue corresponding to amino acid residue 161 of SEQ ID NO: 258. In some embodiments, the polypeptide has an A, N, D, R, C, Q, G, H, I, L, K, M, F, P, S, T, W, Y, or V at an amino acid residue corresponding to amino acid residue 171 of SEQ ID NO: 258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an isoleucine (I) at an amino acid residue corresponding to amino acid residue 139 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO: 258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an A, N, D, R, C, E, Q, G, I, leucine (L), K, M, F, P, S, T, W, Y, or V at an amino acid residue corresponding to amino acid residue 161 of SEQ ID NO: 258; an N at an amino acid residue corresponding to amino acid residue 200 of SEQ ID NO: 258, a K at an amino acid residue corresponding to amino acid residue 306 of SEQ ID NO: 258, an F at an amino acid residue corresponding to amino acid residue 313 of SEQ ID NO: 258, a P at an amino acid residue corresponding to amino acid residue 330 of SEQ ID NO: 258, and a W at an amino acid residue corresponding to amino acid residue 603 of SEQ ID NO: 258. In some embodiments, the polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: a T at an amino acid residue corresponding to amino acid residue 161 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an A, N, D, R, C, Q, G, H, I, L, K, M, F, P, S, T, W, Y, or V at an amino acid residue corresponding to amino acid residue 171 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO: 258, has: a Y at an amino acid residue corresponding to amino acid residue 171 of SEQ ID NO: 258; an N at an amino acid residue corresponding to amino acid residue 200 of SEQ ID NO: 258, a K at an amino acid residue corresponding to amino acid residue 306 of SEQ ID NO: 258, an F at an amino acid residue corresponding to amino acid residue 313 of SEQ ID NO: 258, a P at an amino acid residue corresponding to amino acid residue 330 of SEQ ID NO: 258, and a W at an amino acid residue corresponding to amino acid residue 603 of SEQ ID NO: 258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: a L at an amino acid residue corresponding to amino acid residue 223 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an H at an amino acid residue corresponding to amino acid residue 287 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an R at an amino acid residue corresponding to amino acid residue 289 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an R at an amino acid residue corresponding to amino acid residue 289 of SEQ ID NO:258; a G at an amino acid residue corresponding to amino acid residue 241 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: a G at an amino acid residue corresponding to amino acid residue 241 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an R or S at an amino acid residue corresponding to amino acid residue 236 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide of the present invention comprises an amino acid sequence having at least 70% sequence identity to SEQ ID NO: 258 when the amino acid sequence is optimally aligned thereto, wherein the amino acid sequence, when optimally aligned to SEQ ID NO:258, has: a S at an amino acid residue corresponding to amino acid residue 236 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO: 258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: a S at an amino acid residue corresponding to amino acid residue 236 of SEQ ID NO:258; a G at an amino acid residue corresponding to amino acid residue 241 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: a S at an amino acid residue corresponding to amino acid residue 236 of SEQ ID NO:258; an R at an amino acid residue corresponding to amino acid residue 289 of SEQ ID NO:258; a G at an amino acid residue corresponding to amino acid residue 241 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258.

In some embodiments, a polypeptide of the present invention comprises an amino acid sequence having at least 70% sequence identity to SEQ ID NO:258 when the amino acid sequence is optimally aligned thereto, wherein the amino acid sequence, when optimally aligned to SEQ ID NO:258, has: an H at an amino acid residue corresponding to amino acid residue 252 of SEQ ID NO:258; an F at an amino acid residue corresponding to amino acid residue 325 of SEQ ID NO:258; an A at an amino acid residue corresponding to amino acid residue 332 of SEQ ID NO:258; Y at an amino acid residue corresponding to amino acid residue 335 of SEQ ID NO:258; an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and a W at an amino acid residue corresponding to amino acid residue 603 of SEQ ID NO:258. In some embodiments, the polypeptide further comprises an A, N, D, R, C, E, Q, G, H, I, L, K, M, F, P, S, T, W, or Y at an amino acid residue corresponding to amino acid residue 223 of SEQ ID NO:258, optionally an A or L at an amino acid residue corresponding to amino acid residue 223 of SEQ ID NO:258. In some embodiments, the polypeptide is not mutated at an amino acid residue corresponding to amino acid residue 223 of SEQ ID NO:258. In some embodiments, a polypeptide of the present invention has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524. In some embodiments, a polypeptide of the present invention comprises an amino acid sequence of one or more of SEQ ID NOs: 264-271, 273-283, 303-318, 320-325, 327-329, 331, 333, and 514-524.

A polypeptide of the present invention may generate DNA (e.g., cDNA) from RNA. In some embodiments, a polypeptide of the present invention has activity as an RNA-dependent DNA polymerase and/or the polypeptide generates DNA by polymerizing DNA from one end (e.g., from the 3′end) of a DNA primer and/or RNA primer. In some embodiments, a polypeptide of the present invention may be a monomer or a homodimer and/or may generate DNA from RNA as a monomer or as a homodimer. In some embodiments, a polypeptide of the present invention has reverse transcriptase activity. Activity of a polypeptide of the present invention may be measured by polymerase activity using methods known in the art. Exemplary assays for measuring and/or determining polymerase activity include, but are not limited to, measuring incorporation of a labeled nucleotide, e.g., a radiolabeled, chemiluminescent, and/or colorimetrically (e.g., fluorescently) labeled nucleotide, and/or synthetic nucleoside analogues (e.g., 4-thiouridine and/or bromodeoxyuridine). In some embodiments, radiolabeling is used to measure and/or determine polymerase activity (e.g., measuring the quantity of a radiolabeled nucleotide included in a polymerized nucleic acid). In some embodiments, a primer extension assay is used to measure and/or determine polymerase activity, optionally wherein the primer extension assay uses a labeled primer (e.g., a fluorescently labeled primer such as a Cy3 or Cy5 labeled primer) to determine the rate of synthesis. In some embodiments, a cleavage assay is used to measure and/or determine polymerase activity (e.g., RNase activity).

In some embodiments, the activity of a polypeptide of the present invention is measured by the number of nucleotides generated (e.g., polymerized) during one cell division and/or in about 20 minutes. Cell division and/or the time period for one cell division can be readily determined by one of skill in the art. In some embodiments, the cell division is a cell division for a bacterial cell, a human cell, or a plant cell. In some embodiments, the time of one cell division is about 18 to about 22 minutes, about 19 to about 21 minutes, or about 20 minutes. In some embodiments, a polypeptide of the present invention generates (e.g., polymerizes) at least 18, 19, 20, 21, 22, 23, 24, 25 or more nucleotides during one cell division and/or in about 20 minutes, optionally at a temperature of about 10° C., 15° C., 20° C., 25° C., 30° C., 35° C., 40° C., 45° C., 50° C., 55° C., 60° C., 65° C., 70° C., 75° C., or 80° C. Polypeptide activity may be measured by the rate of nucleotides generated (e.g., polymerized) in a period of time. In some embodiments, a polypeptide of the present invention polymerizes at least 18, 19, 20, 21, 22, 23, 24, or 25 nucleotides in 20 minutes. In some embodiments, a polypeptide of the present invention polymerizes at least 0.9, 0.95, 1.0, 1.05, 1.1, 1.15, 1.20, 1.25 or more nucleotides per minute at a physiologically relevant temperature. In some embodiments, a polypeptide of the present invention generates nucleotides at a rate of at least 0.9, 0.95, 1.0, 1.05, 1.1, 1.15, 1.20, or 1.25 nucleotides per minute at a temperature of about 10° C., 15° C., 20° C., 25° C., 30° C., 35° C., 40° C., 45° C., 50° C., 55° C., 60° C., 65° C., 70° C., 75° C., or 80° C. In some embodiments, a polypeptide of the present invention generates (e.g., polymerizes) at least 23 nucleotides during one cell division and/or in about 20 minutes, optionally at a temperature of about 10° C., 15° C., 20° C., 25° C., 30° C., 35° C., 40° C., 45° C., 50° C., 55° C., 60° C., 65° C., 70° C., 75° C., or 80° C.

In some embodiments, a polypeptide of the present invention generates DNA from RNA at a temperature in a range from about 10° C., 15° C., 20° C., 25° C., 30° C., 35° C., 40° C., 45° C. or 50° C. to about 55° C., 60° C., 65° C., 70° C., 75° C., or 80° C. In some embodiments, a polypeptide of the present invention generates DNA from RNA at a temperature of about 50° C. or less such as at a temperature of about 10° C., 15° C., 20° C., 25° C., 30° C., 35° C., 40° C., or 45° C. In some embodiments, a polypeptide of the present invention generates DNA from RNA at a temperature in a range of about 10° C. to about 40° C., about 15° C. to about 35° C., about 15° C. to about 40° C., about 15° C. to about 50° C., about 18° C. to about 30° C., about 18° C. to about 25° C., about 20° C. to about 25° C., about 20° C. to about 80° C., about 20° C. to about 50° C., about 20° C. to about 45° C., about 30° C. to about 50° C., about 30° C. to about 45° C., about 30° C. to about 40° C., about 20° C. to about 22° C., about 18° C., 19° C., 20° C., 21° C., 22° C., 23° C., 24° C., or 25° C., or about room temperature.

As used herein, “processivity” refers to the number of nucleotides generated (e.g., synthesized) in a single binding event of a polypeptide of the present invention. Processivity may be measured in vitro and/or in vivo using methods known in the art. In some embodiments, processivity may be measured by the number of bases generated over a period of time per unit of enzyme (e.g., polypeptide of the present invention), wherein 1 unit of enzyme is the amount of enzyme that will incorporate 1 nmol of dTTP into acid-insoluble material in a total reaction volume of 50 μl in 10 minutes at 37° C. using poly(rA)·oligo(dT) as the template primer with 50 mM Tris-HCl (pH 8.3), 6 mM MgCl2, 10 mM dithiothreitol, 0.5 mM [3H]-dTTP and 0.4 mM poly(rA)·oligo(dT) 12-18. In some embodiments, a polypeptide of the present invention has a processivity of at least about 100, 250, 500, 1000, 1500, or 2000 nucleotides or more, e.g. about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2500, 3000, 3500, 4000, 4500, or 5000 nucleotides or more. In some embodiments, a polypeptide of the present invention has a processivity of about 100 to about 600 nucleotides, about 200 to about 500 nucleotides, or at least about 300 nucleotides.

A polypeptide of the present invention may have a processivity that is increased compared to the processivity of a control reverse transcriptase. A “control reverse transcriptase” or “control RT” as used herein refers to a naturally occurring reverse transcriptase or commercially available reverse transcriptase. Exemplary control RTs include, but are not limited to, Moloney Murine Leukemia Virus Reverse Transcriptase (MMLV-RT or M-MuLV-RT), mutated MMLV (e.g., 5M-MMLV), Avian Myeloblastosis Virus Reverse Transcriptase (AMV-RT), Human Immunodeficiency Virus Reverse Transcriptase (HIV-RT). In some embodiments, the control RT may be MMLV and/or 5M-MMLV. In some embodiments, a control RT may have a sequence of one of SEQ ID NOs: 122-124, 132-169, 208-251, and 258 and/or is encoded by a nucleotide having a sequence of one of SEQ ID NOs: 120, 121, 125-131 170-207, 252-257, and 334. Processivity for a polypeptide of the present invention and for control RT may be measured by using one or more sequences under the same reaction conditions (e.g., same time, temperature, concentration, sequence identity, modification, etc.). In some embodiments, a polypeptide of the present invention may have a processivity that is reduced compared to (e.g., lower than) the processivity of a control RT and/or may have a processivity that is faster than a DNA repair enzyme's ability to correct a modification such that the polypeptide can out-run the DNA repair enzyme. In some embodiments, a polypeptide of the present invention has a processivity at a first temperature that is equal to or better than the processivity of a control reverse transcriptase at a second temperature, wherein the first temperature is lower than the second temperature. For example, a polypeptide of the present invention may have a processivity at room temperature that is equal to or better than the processivity of a control reverse transcriptase such as 5M-MMLV at a temperature of greater than room temperature, for example, about 42° C. or about 55° C. In some embodiments, the processivity of a polypeptide of the present invention is within ±about 5%, about 10%, about 15%, about 20%, or about 25% of the processivity of the DNA polymerase in the cell. In some embodiments, the processivity of the polypeptide of the present invention is within about 5%, about 10%, about 15%, about 20% or about 25% of the rate of one or more replicative polymerases of the cell, one or more repair polymerases of the cell, or a combination thereof.

In some embodiments, a polypeptide of the present invention is devoid of at least a portion of an RNAseH domain. RNAseH domains are conserved across viral RTs and are structurally similar to RNaseH domains in Escherichia coli, Bacillus halodurans and human RNase H1. See, e.g., Champoux et al., FEBS Journal, 276:6, 1506-1516 (2009). Enzymatic activity is conferred by the DEDD sequence motif, a conserved sequence composed of aspartate and glutamate residues, which, in HIV-1 RNase H, are Asp443, Glu478, Asp498 and Asp549, with the corresponding active site amino acids in the M-MLV enzyme of Asp524, Glu562, Asp583 and Asp653.

In some embodiments, a polypeptide of the present invention has reduced (e.g., about 90%, 80%, 70%, 60%, 50%, 40%, 30%, 20%, 15%, 10%, 5%, or less) or no ribonuclease (RNase) activity. In some embodiments, a polypeptide of the present invention has reduced (e.g., about 90%, 80%, 70%, 60%, 50%, 40%, 30%, 20%, 15%, 10%, 5%, or less) or no ribonuclease (RNase) activity compared to a control RT. In some embodiments, the control reverse transcriptase may be an RT with high RNase activity, for example, avian myeloblastosis virus (AMV) or medium RNasH activity, for example, MMLV-RT and/or 5M-MMLV.

In some embodiments, a polypeptide of the present invention generates and/or is capable of generating DNA from RNA in two or more different species (e.g., 3, 4, 5, 6, 7, 8, 9, or 10, or more different species). In some embodiments, a polypeptide of the present invention generates DNA from RNA in two or more different species and has a processivity of at least about 100, 200, 300, 400, 500, 600, 700, 800, 900, 1,000, 1100, 1200, 1300, 1400, 1500, 1600, 1700, 1800, 1900, or 2,000 nucleotides or more, optionally at a temperature in a range of about 25° C., 30° C., 35° C., 40° C., 45° C. or 50° C. to about 55° C., 60° C., 65° C., 70° C., 75° C., or 80° C. In some embodiments, a polypeptide of the present invention generates DNA from RNA in two or more different species at a temperature in a range of about 20° C., 25° C., 30° C., 35° C., 40° C., 45° C. or 50° C. to about 55° C., 60° C., 65° C., 70° C., 75° C., or 80° C. for each of the two or more different species. In some embodiments, a polypeptide of the present invention generates DNA from RNA in two or more different species and has a processivity of at least about 100, 200, 300, 400, 500, 600, 700, 800, 900, 1,000, 1100, 1200, 1300, 1400, 1500, 1600, 1700, 1800, 1900, or 2,000 nucleotides or more, optionally at a temperature of less than 50° C. (e.g., about 20° C., 25° C., 30° C., 35° C., 40° C., or 45° C.) or at a temperature in a range of about 20° C., 25° C., or 30° C. to about 35° C., 40° C., or 45° C.

A polypeptide of the present invention may generate DNA from RNA in a prokaryote or a eukaryote. In some embodiments, a polypeptide of the present invention generates DNA from RNA in a plant and/or an animal. In some embodiments, a polypeptide of the present invention generates DNA from RNA in corn, soy, canola, wheat, rice, cotton, sugarcane, sugar beet, barley, oats, alfalfa, sunflower, safflower, oil palm, sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, blackberry, raspberry, black raspberry, and/or a Brassica spp.

A polypeptide of the present invention may be a fusion protein. In some embodiments, a polypeptide of the present invention is fused (e.g., directly or indirectly such as via a linker) to a CRISPR-Cas effector protein or a portion thereof.

A polypeptide of the present invention (optionally in a complex of the present invention) may have an editing efficiency (e.g., indel percentage) that is the same as or greater than (e.g., increased compared to) a control reverse transcriptase (optionally in a complex with comparable components to those in the complex with the polypeptide of the present invention). In some embodiments, a polypeptide of the present invention has an editing efficiency that is the same as or greater than a control RT when performed at a temperature of less than 50° C. such as about 45° C., about 42° C., about 37° C., about 30° C., about 25° C., or about room temperature.

In some embodiments, a polypeptide of the present invention, which has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524, has at least about 70%, 75%, 80%, 85%, 90%, 95%, 96%, 97%, 98%, 99%, or more sequence identity to SEQ ID NO:258. In some embodiments, a polypeptide of the present invention (e.g., having at least about 70% sequence identity to one or more of SEQ ID NOs: 264-271, 273-283, 303-318, 320-325, 327-329, 331, 333, and 514-524) has at least about 90%, 91%, 92%, 93%, 94%, 95%, 96%, 97%, 98%, 99%, 99.5%, or more sequence identity to SEQ ID NO: 258. In some embodiments, a polypeptide of the present invention (e.g., having at least about 70% sequence identity to one or more of SEQ ID NOs: 264-271, 273-283, 303-318, 320-325, 327-329, 331, 333, and 514-524) has at least about 90%, 91%, 92%, 93%, 94%, 95%, 96%, 97%, 98%, 99%, 99.5%, or more sequence identity to SEQ ID NO:258 and has 1, 2, 3, 4, 5, 6, 7, 8, or more mutation(s) (e.g., one or more non-natural mutation(s)) relative to the sequence of SEQ ID NO:258, optionally when optimally aligned thereto.

According to some embodiments of the present invention, a complex is provided that comprises a CRISPR-Cas effector protein; a polypeptide of the present invention; and an extended guide nucleic acid. In some embodiments, the polypeptide has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524. In some embodiments, the complex comprises a CRISPR-Cas effector protein; a polypeptide comprising a sequence of one or more of SEQ ID NOs: 264-271, 273-283, 303-318, 320-325, 327-329, 331, 333, and 514-524; and an extended guide nucleic acid. In some embodiments, the CRISPR-Cas effector protein is a Type V CRISPR-Cas effector protein or a Type II CRISPR-Cas effector protein. The CRISPR-Cas protein may be a fusion protein. In some embodiments, the CRISPR-Cas effector protein may comprise a CRISPR-Cas effector polypeptide fused to a peptide tag. In some embodiments, the CRISPR-Cas effector protein may comprise a CRISPR-Cas effector polypeptide fused to an affinity polypeptide that is capable of binding a peptide tag. In some embodiments, the CRISPR-Cas effector protein may comprise a CRISPR-Cas effector polypeptide fused to an affinity polypeptide that is capable of binding an RNA recruiting motif. In some embodiments, when the CRISPR-Cas protein comprises a peptide tag, the polypeptide of the present invention comprises an affinity polypeptide that is capable of binding the peptide tag. In some embodiments, when the CRISPR-Cas protein comprises an affinity polypeptide, the polypeptide of the present invention comprises a peptide tag that is capable of binding the affinity polypeptide. In some embodiments, the polypeptide of the present invention may be fused to an affinity polypeptide that is capable of binding an RNA recruiting polypeptide. A complex of the present invention may further comprise a guide nucleic acid that is optionally devoid of a reverse transcriptase template. In some embodiments, the guide nucleic acid is directed to a different target nucleic acid than the extended guide nucleic acid.

A nucleic acid molecule may encode a polypeptide of the present invention, which may be present in an expression cassette and/or vector. A polynucleotide and/or recombinant nucleic acid construct of this invention can be codon optimized for expression. In some embodiments, a polynucleotide, nucleic acid construct, expression cassette, and/or vector of the present invention (e.g., that comprises/encodes a polypeptide of the present invention, a nucleic acid binding polypeptide (e.g., a DNA binding domain such as a sequence-specific DNA binding domain from a polynucleotide-guided endonuclease such as a CRISPR-Cas effector protein), and/or an extended guide nucleic acid) may be codon optimized for expression in an organism (e.g., an animal, a plant, a fungus, an archaeon, or a bacterium). In some embodiments, an expression cassette and/or vector of the present invention comprises a polynucleotide that encodes a promoter sequence and a polynucleotide that encodes a polypeptide of the present invention. In some embodiments, the polypeptide has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524. The polynucleotide encoding the polypeptide of the present invention may be codon-optimized for expression in a particular organism (e.g., a human or plant). In some embodiments, an expression cassette and/or vector of the present invention comprises a polynucleotide that encodes a promoter sequence and a polynucleotide that has at least about 70%, 75%, 80%, 85%, 90%, 95%, 96%, 97%, 98%, 99%, or more sequence identity to one or more of SEQ ID NOs: 340-347, 349-359, 379-394, 396-401, 403-405, 407, and 409. In some embodiments, an expression cassette and/or vector of the present invention comprises a polynucleotide having a nucleotide sequence of one or more of SEQ ID NOs: 340-347, 349-359, 379-394, 396-401, 403-405, 407, and 409. In some embodiments, the expression cassette and/or vector further comprises a polynucleotide encoding a CRISPR-Cas effector protein that is codon-optimized for expression in the organism. In some embodiments, the organism is an animal (e.g., a human), a plant, a fungus, an archaeon, or a bacterium. In some embodiments, a polynucleotide encoding a polypeptide of the present invention comprises an intron with at least about 70%, 75%, 80%, 85%, 90%, 95%, 96%, 97%, 98%, 99%, or 100% sequence identity to SEQ ID NO: 512. In some embodiments, a polynucleotide encoding a polypeptide of the present invention comprises an intron having the sequence of SEQ ID NO:512.

In some embodiments, an expression cassette and/or vector of the present invention comprises an intron. In some embodiments, an expression cassette and/or vector of the present invention comprises a polynucleotide encoding a polypeptide of the present invention and an intron having a polynucleotide sequence with at least about 70%, 75%, 80%, 85%, 90%, 95%, 96%, 97%, 98%, 99%, or 100% sequence identity to SEQ ID NO:512. In some embodiments, an expression cassette and/or vector of the present invention comprises a polynucleotide encoding a polypeptide of the present invention and an intron having a polynucleotide sequence of SEQ ID NO:512. In some embodiments, an expression cassette and/or vector of the present invention comprising a polynucleotide encoding a polypeptide of the present invention and an intron having at least about 70%, 75%, 80%, 85%, 90%, 95%, 96%, 97%, 98%, 99%, or 100% sequence identity to SEQ ID NO:512 has increased expression (e.g., about 5%, 10%, 15%, 20%, 25%, 30%, 35%, 40%, 45%, 50%, 55%, 60%, 65%, 70%, 75%, 80%, 85%, 90%, 95%, 100%, or more increased expression) of said polypeptide from said expression cassette and/or vector compared to an expression cassette and/or vector that is devoid of said intron. In some embodiments, an expression cassette and/or vector of the present invention comprising a polynucleotide encoding a polypeptide of the present invention and an intron having at least about 70%, 75%, 80%, 85%, 90%, 95%, 96%, 97%, 98%, 99%, or 100% sequence identity to SEQ ID NO:512 has increased transformation efficiency (e.g., about 5%, 10%, 15%, 20%, 25%, 30%, 35%, 40%, 45%, 50%, 55%, 60%, 65%, 70%, 75%, 80%, 85%, 90%, 95%, 100%, or more increased transformation efficiency) and/or reduced transformation-associated toxicity (e.g., about 5%, 10%, 15%, 20%, 25%, 30%, 35%, 40%, 45%, 50%, 55%, 60%, 65%, 70%, 75%, 80%, 85%, 90%, or 95% reduced transformation-associated toxicity) when compared to an expression cassette and/or vector that is devoid of said intron. Transformation-associated toxicity, as used herein, is intended to mean the amount cell death and/or cytotoxic response that occurs in a population of cells during the transformation process. Transformation efficiency, as used herein, is intended to mean the number of cells that become transformed in a population of cells during the transformation process. In some embodiments, the transformation efficiency is measured based on the concentration of polynucleotide, polypeptide, and/or ribonucleoprotein used in the transformation process (e.g., high transformation efficiency uses less concentration of polynucleotide, polypeptide, and/or ribonucleoprotein; and low transformation efficiency uses more concentration of polynucleotide, polypeptide, and/or ribonucleoprotein).

Methods of modifying a target nucleic acid in a cell are provided, and may comprise introducing an expression cassette and/or vector of the present invention into the cell to provide a modified target nucleic acid. In some embodiments, the cell is a plant cell and the method further comprises regenerating the plant cell comprising the modified target nucleic acid to produce a plant comprising the modified target nucleic acid. In some embodiments, the introducing of the expression cassette is carried out at a temperature of about 20° C. to about 42° C.

Methods for producing a polypeptide of the present invention are provided herein and may comprise culturing a cell or a plurality of cells that have been transformed with a nucleic acid encoding a polypeptide of the present invention; and isolating the polypeptide of the present invention to thereby produce the polypeptide. In some embodiments, the step of isolating is performed by dialysis, centrifugation, column purification, and/or the like.

Methods for performing reverse transcription are provided herein and may comprise contacting a target nucleic acid with a polypeptide of the present invention. In some embodiments, the polypeptide has at least about 70%, 75%, 80%, 85%, 90%, 95%, 96%, 97%, 98%, 99%, or 100% sequence identity to one or more of SEQ ID NOs: 264-271, 273-283, 303-318, 320-325, 327-329, 331, 333, and 514-524. In some embodiments, a method of the present invention may comprise comparing an activity (e.g., a reverse transcriptase activity), precise editing percentage, and/or indel percentage of a polypeptide of the present invention (e.g., having at least about 70%, 75%, 80%, 85%, 90%, 95%, 96%, 97%, 98%, 99%, or more sequence identity to one or more of SEQ ID NOs: 264-271, 273-283, 303-318, 320-325, 327-329, 331, 333, and 514-524) following contact to a target nucleic acid to the same activity, a precise editing percentage, and/or an indel percentage of a protein having a sequence of one of SEQ ID NO:122-124, 132-169, 208-251, and 258 following contact of the protein to the same target nucleic acid. The polypeptide of the present invention may reverse transcribe the target nucleic acid to provide a DNA (e.g., a cDNA). In some embodiments, the target nucleic acid is in a plant and/or in a plant cell that includes a cell wall. In some embodiments, the target nucleic acid is in a mammal and/or is in a mammalian cell.

Methods of modifying a target nucleic acid are provided herein and may comprise contacting the target nucleic acid with a CRISPR-Cas effector protein, a polypeptide of the present invention, and an extended guide nucleic acid, optionally wherein the polypeptide of the present invention has at least about 70%, 75%, 80%, 85%, 90%, 95%, 96%, 97%, 98%, 99%, or more sequence identity to one or more of SEQ ID NOs: 264-271, 273-283, 303-318, 320-325, 327-329, 331, 333, and 514-524. In some embodiments, the target nucleic acid is in a plant and/or in a plant cell that includes a cell wall. In some embodiments, the method of modifying the target nucleic acid is a templated editing method.

A method of the present invention may comprise contacting the target nucleic acid with an extended guide nucleic acid. In some embodiments, the extended guide nucleic acid comprises a primer binding site and the target nucleic acid is double stranded and comprises a first strand and a second strand. In some embodiments, the primer binding site of the extended guide nucleic acid binds to the first strand or to the second strand of the target nucleic acid. In some embodiments, the second strand is the non-target strand of the target nucleic acid. In some embodiments, the target nucleic acid is double stranded and comprises a first strand and a second strand and the primer binding site binds to the first strand of the target nucleic acid. The first strand may be the target strand of the target nucleic acid and/or the CRISPR-Cas effector protein is recruited to the first strand. In some embodiments, the target nucleic acid is double stranded and comprises a first strand and a second strand and the primer binding site of the extended guide nucleic acid binds to the second strand of the target nucleic acid. In some embodiments, the second strand is the non-target strand of the target nucleic acid and/or the CRISPR-Cas effector protein is recruited to the second strand. In some embodiments, the CRISPR-Cas effector protein is a double stranded nuclease that cuts the first strand and the second strand of the target nucleic acid resulting in a double stranded break. In some embodiments, the CRISPR-Cas effector protein, the polypeptide of the present invention, and the extended guide nucleic acid form a complex or are comprised in a complex.

A method of the present invention may comprise contacting a target nucleic acid with and/or introducing into a cell an extended guide nucleic acid that comprises (i) a CRISPR nucleic acid and/or a CRISPR nucleic acid and a tracr nucleic acid; and (ii) an extended portion comprising a primer binding site and a reverse transcriptase template (RT template). In some embodiments, the extended portion of the extended guide nucleic acid is fused to either the 5′ end or 3′ end of the CRISPR nucleic acid (e.g., 5′ to 3′: repeat-spacer-extended portion, or extended portion-repeat-spacer) and/or to the 5′ or 3′ end of the tracr nucleic acid. In some embodiments, the extended portion of the extended guide nucleic acid comprises, 5′ to 3′, an RT template and a primer binding site. In some embodiments, the extended portion of the extended guide nucleic acid is located 5′ of the crRNA.

In some embodiments, the primer binding site has a length of about one nucleotide to about 100 nucleotides. In some embodiments, the primer binding site is at least 45 nucleotides in length or about 45 nucleotides to about 100 nucleotides, e.g., 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the RT template has a length of about one to about 100 nucleotides, or the RT template can have a length of about 40 nucleotides or less, e.g., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide.

In some embodiments, the extended portion of the extended guide nucleic acid is linked to the CRISPR nucleic acid and/or the tracrRNA via a linker. The linker may be about 1 to about 100 nucleotides in length, e.g., 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, in a method of the present invention, a CRISPR-Cas effector protein is a fusion protein and/or a polypeptide of the present invention is a fusion protein. In some embodiments, the CRISPR-Cas effector protein, the polypeptide of the present invention, and/or the extended guide nucleic acid is fused to one or more components that recruit the polypeptide to the CRISPR-Cas effector protein. In some embodiments, the CRISPR-Cas effector protein is a Type V CRISPR-Cas effector fusion protein comprising a Type V CRISPR-Cas effector polypeptide fused, e.g., linked, to a peptide tag, for example, an epitope or a multimerized epitope, and the polypeptide of the present invention is a reverse transcriptase fusion protein comprising the polypeptide fused, e.g., linked, to an affinity polypeptide that binds to the peptide tag. In some embodiments, the target nucleic acid is contacted with two or more reverse transcriptase fusion proteins. In some embodiments, the CRISPR-Cas effector protein is a Type II CRISPR-Cas effector fusion protein comprising a Type II CRISPR-Cas effector polypeptide fused, e.g., linked, to a peptide tag, for example, an epitope or a multimerized epitope, and the polypeptide of the present invention is a reverse transcriptase fusion protein comprising the polypeptide fused (linked) to an affinity polypeptide that binds to the peptide tag. In some embodiments, the target nucleic acid is contacted with two or more reverse transcriptase fusion proteins.

In some embodiments, the extended guide nucleic acid is linked to an RNA recruiting motif, and a polypeptide of the present invention is a reverse transcriptase fusion protein comprising the polypeptide fused, e.g., linked, to an affinity polypeptide that binds to the RNA recruiting motif. In some embodiments, the target nucleic acid is contacted with two or more reverse transcriptase fusion proteins. In some embodiments, the extended guide nucleic acid, e.g., extended guide RNA, is linked to two or more RNA recruiting motifs, optionally wherein the two or more RNA recruiting motifs are the same RNA recruiting motif or different RNA recruiting motifs. In some embodiments, at least one of the two or more RNA recruiting motifs is located on the 3′ end of the extended portion of the extended guide nucleic acid or is embedded in the extended portion.

A method of the present invention may comprise contacting the target nucleic acid with a Dna2 polypeptide and/or a 5′ flap endonuclease (FEN). In some embodiments, the FEN and/or Dna2 polypeptide is overexpressed (e.g., overexpressed in the presence of the target nucleic acid). In some embodiments, the FEN is a fusion protein comprising an FEN domain fused to the CRISPR-Cas effector protein and/or wherein the Dna2 polypeptide is a fusion protein comprising an Dna2 domain fused to the CRISPR-Cas effector protein. In some embodiments, the CRISPR-Cas effector protein is a first CRISPR-Cas effector protein and the method includes a step of contacting the target nucleic acid with a second CRISPR-Cas effector protein. In some embodiments, the first CRISPR-Cas effector protein nicks or cuts a first site on the first strand of a double stranded target nucleic acid. In some embodiments, the nicking or cut site is located about 10 to about 125 base pairs upstream or downstream, e.g., 5′ or 3′, from a second site on the second strand of the target nucleic acid that has been nicked by a second CRISPR-Cas effector protein.

In some embodiments, a method of the present invention has increased efficiency in modifying a target nucleic acid compared to the efficiency of a control method (e.g., a method that uses a control RT and is performed under the same conditions). A method of the present invention may generate increased indels and/or increased levels of modification (e.g., precise modifications) compared to a control method.

In some embodiments, a complex and/or method of the present invention may be a complex and/or method as described in U.S. Patent Application Publication No. 2021/0130835 and/or in U.S. Patent Application Publication No. 2022/0145334, the contents of each of which are incorporated herein by reference in their entirety, but where the reverse transcriptase is a polypeptide of the present invention.

In some embodiments, an editing system of the present invention is used in prime editing. “Prime editing” and grammatical variants thereof as used herein refer to a nucleic acid editing technology that uses a Cas9 nickase domain fused to a reverse transcriptase and modifies a target nucleic acid without a double strand break or a donor DNA template. In Prime editing, the Cas9 nickase domain cuts the non-complementary strand of DNA upstream of the PAM site, thereby providing a 3′ flap that is extended with the extension including a modification. Further details on Prime editing can be found in Anzalone et al. (2019) Nature 576, 149-157 and/or U.S. Patent Application Publication No. 2021/0147862, the contents of each of which are incorporated herein by reference in their entirety.

In some embodiments, an editing system of the present invention utilizes the Redraw editing system. Further details on the Redraw editing system can be found in U.S. Patent Application Publication No. 2021/0130835 and/or in U.S. Patent Application Publication No. 2022/0145334, the contents of each of which are incorporated herein by reference in their entirety.

As described herein, polypeptides of the present invention, nucleic acids, expression cassettes, and/or vectors of the present invention may be codon optimized for expression in an organism. An organism useful with this invention may be any organism or cell thereof for which nucleic acid modification may be useful. An organism can include, but is not limited to, any animal (e.g., a mammal), any plant, any fungus, any archaeon, or any bacterium. In some embodiments, the organism may be a plant or cell thereof. In some embodiments, the organism is an animal such as a mammal (e.g., a human).

The target nucleic acid may be a genomic sequence from any organism (e.g., eukaryote such as a mammal or a plant). In some embodiments, the target nucleic acid is a genomic sequence from a model organism such as, but not limited to, Escherichia coli, an immortalized human cell line (e.g., HEK293, HeLa, etc.), Caenorhabditis elegans, and/or Drosophila Melanogaster. In some embodiments, the target nucleic acid is a genomic sequence from a non-model organism. Exemplary non-model organisms include, but are not limited to crop plants (e.g., fruit crop plants, vegetable crop plants, and/or field crop plants) and/or animals such as humans, primates and/or mice. In some embodiments, the non-model organism is a crop plant such as corn, soybean, wheat, or canola. In some embodiments, the non-model organism is an animal for testing and/or use of a human therapeutic.

A target nucleic acid of any plant or plant part may be modified using the nucleic acid constructs of the invention. Any plant (or groupings of plants, for example, into a genus or higher order classification) may be modified using a polypeptide of the present invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry.

In some embodiments, the invention provides cells (e.g., plant cells, animal cells, bacterial cells, archacon cells, and the like) comprising the polypeptides, polynucleotides, nucleic acid constructs, expression cassettes or vectors of the invention.

The present invention further comprises a kit or kits to carry out the methods of this invention. A kit of this invention can comprise reagents, buffers, and apparatus for mixing, measuring, sorting, labeling, etc., as well as instructions and the like as would be appropriate for modifying a target nucleic acid.

In some embodiments, the invention provides a kit for comprising one or more polypeptides of the present invention, nucleic acid constructs of the invention, and/or expression cassettes and/or vectors and/or cells comprising the same as described herein, with optional instructions for the use thereof. In some embodiments, a kit may further comprise a CRISPR-Cas guide nucleic acid (corresponding to a CRISPR-Cas effector protein as provided herein, which may be encoded by a polynucleotide) and/or expression cassettes and/or vectors and or cells comprising the same. In some embodiments, a guide nucleic acid may be provided on the same expression cassette and/or vector as one or more nucleic acid constructs of the invention. In some embodiments, the guide nucleic acid may be provided on a separate expression cassette or vector from that comprising the one or more nucleic acid constructs of the invention.

Accordingly, in some embodiments, kits are provided comprising a nucleic acid construct comprising (a) a polynucleotide(s) as provided herein and (b) a promoter that drives expression of the polynucleotide(s) of (a). In some embodiments, the kit may further comprise a nucleic acid construct encoding a guide nucleic acid, wherein the construct comprises a cloning site for cloning of a nucleic acid sequence identical or complementary to a target nucleic acid sequence into backbone of the guide nucleic acid.

In some embodiments, the nucleic acid construct of the invention may be an mRNA that may encode one or more introns within the encoded polynucleotide(s). In some embodiments, the nucleic acid constructs of the invention, and/or an expression cassettes and/or vectors comprising the same, may further encode one or more selectable markers useful for identifying transformants (e.g., a nucleic acid encoding an antibiotic resistance gene, herbicide resistance gene, and the like).

A polypeptide, polynucleotide, nucleic acid construct, expression cassette, vector, composition, kit, system and/or cell of the present invention may comprise all or a portion of a sequence of one or more of SEQ ID NOs: 1-524. In some embodiments, a polypeptide, polynucleotide, nucleic acid construct, expression cassette, vector, composition, kit, system and/or cell of the present invention may comprise at least about 20%, 25%, 30%, 35%, 40%, 45%, 50%, 55%, 60%, 65%, 70%, 75%, 80%, 85%, 90%, 95%, 96%, 97%, 98%, 99%, or more consecutive amino acids of a sequence of one or more of SEQ ID NOs: 1-524.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1: Variants of MMLV-RT (5M)

Protein domains of MMLV-RT (5M) based upon the crystal structure of the protein have been identified along with the location of certain amino acid residues and are outlined in Table 1.

TABLE 1
Domains of MMLV-RT(5M)

| Amino acid residue in | Position in domain based |
|---|---|
| Reverse Transcriptase | upon crystal structure |
| Proline 65 | Fingers |
| Methionine 66 | Fingers |
| Serine 67 | Fingers |
| Glutamic Acid 69 | Fingers |
| Cystein 90 | Fingers |
| Glutamine 91 | Fingers |
| Lysine 102 | Fingers |
| Lysine 103 | Fingers |
| Proline 104 | Fingers |
| Arginine 116 | Fingers |
| Lysine 120 | Fingers |
| Histidine 126 | Palm |
| Proline 130 | Palm |
| Leucine 139 | Palm |
| Aspartic Acid 150 | Palm |
| Lysine 152 | Palm |
| Aspartic Acid 153 | Palm |
| Phenylalanine 155 | Palm |
| Histidine 161 | Fingers |
| Phenylalanine 168 | Fingers |
| Phenylalanine 170 | Fingers |
| Glutamic Acid 171 | Fingers |
| Tryptophan 172 | Fingers |
| Arginine 173 | Fingers |
| Aspartic Acid 174 | Fingers |
| Methionine 177 | Fingers |
| Tryptophan 185 | Fingers |
| Arginine 187 | Fingers |
| Leucine 188 | Fingers |
| Phenylalanine 192 | Fingers |
| Threonine 197 | Thumb |
| Aspartic Acid 200 | Thumb |
| Glutamic Acid 201 | Thumb |
| Leucine 203 | Thumb |
| Histidine 204 | Thumb |
| Glutamine 213 | Thumb |
| Glutamine 221 | Thumb |
| Tyrosine 222 | Thumb |
| Valine 223 | Thumb |
| Glutamine 237 | Thumb |
| Asparagine 249 | Thumb |
| Lysine 257 | Palm |
| Lysine 258 | Palm |
| Tyrosine 271 | Connection |
| Threonine 287 | Connection |
| Proline 297 | Connection |
| Glutamic Acid 302 | Connection |
| Threonine 306 | Connection |
| Phenylalanine 309 | Connection |
| Tryptophan 313 | Connection |
| Lysine 329 | Connection |
| Threonine 330 | Connection |
| Leucine 333 | Connection |
| Tryptophan 336 | Connection |
| Isoleucine 347 | Connection |
| Tryptophan 388 | Connection |
| Cysteine 409 | Connection |
| Valine 433 | Connection |
| Leucine 435 | Connection |
| Aspartic Acid 449 | Connection |
| Tryptophan 451 | Connection |
| Asparagine 454 | Connection |
| Alanine 481 | RnaseH |
| Aspartic Acid 524 | RnaseH |

Based upon the crystal structure, specific amino acids were identified as possible mutation sites. Tables 2-5 outline the amino acid locations in the appropriate domain which were modified.

TABLE 2
Amino acids for Thumb
Domain of MMLV-RT(5M)
Position

Asparagine 200
Tyrosine 222
Histidine 204
Glutamine 221

TABLE 3
Amino acids for Fingers
Domain of MMLV-RT(5M)
Position

Lysine 102
Lysine 103
Arginine 116
Lysine 120

TABLE 4
Amino acids for Connection
Domain of MMLV-RT(5M)
Position

Arginine 298
Glutamic Acid 302
Phenylalanine 309
Lysine 329

TABLE 5
Amino acids for Palm
Domain of MMLV-RT(5M)
Position

Lysine 152
Lysine 257
Lysine 258
Aspartic Acid 124

### Example 2: Yeast and Human Cell-Based Demonstration of Reverse Transcriptase Variants Activity

A yeast host was used to demonstrate modified reverse transcriptase activity. The host yeast cell line was modified to generate an adenine auxotroph by mutating the ADE2 gene at position Glycine156 (GGA) to the stop codon TAA. This resulted in a yeast strain that requires the addition of adenine to the growth media in order to survive. The adenine auxotroph yeast host was transformed with a variant MMLV-RT (5M) protein or MMLV-RT (5M) (as a control; SEQ ID NO:258) and the guide nucleic acid that directs the modification of the ADE2 gene. Successful editing of the ADE2 gene in the yeast genome results in a yeast cell being able to grow without the addition of adenine to the media. The proteins listed in Table 6a allowed the yeast cell to grow in the absence of adenine. Additional proteins identified are provided in Table 6b.

Reverse transcriptase variants described in Table 6a were also evaluated in human cells to determine editing efficiency.

TABLE 6a
MMLV-RT(5M) Variants Tested in Human Cells (HEK293T).

| | Variant (Mutations | DNA SEQ | Protein SEQ |
|---|---|---|---|
| pWISE | in addition to 5M) | ID NO: | ID NO: |
| pWISE6932 | M66I | 335 | 259 |
| pWISE6933 | M66L | 336 | 260 |
| pWISE6934 | M66V | 337 | 261 |
| pWISE6935 | M66N | 338 | 262 |
| pWISE6936 | M66K | 339 | 263 |
| pWISE6937 | H161Y | 340 | 264 |
| pWISE6938 | H161L | 341 | 265 |
| pWISE6939 | H161A | 342 | 266 |
| pWISE6940 | H161D | 343 | 267 |
| pWISE6941 | H161T | 344 | 268 |
| pWISE6942 | F168V | 345 | 269 |
| pWISE6943 | F168M | 346 | 270 |
| pWISE6944 | F168C | 347 | 271 |
| pWISE6945 | F168H | 348 | 272 |
| pWISE6946 | E171N | 349 | 273 |
| pWISE6947 | E171V | 350 | 274 |
| pWISE6948 | E171D | 351 | 275 |
| pWISE6949 | E171Y | 352 | 276 |
| pWISE6950 | E171F | 353 | 277 |
| pWISE6951 | L139G | 354 | 278 |
| pWISE6952 | L139Y | 355 | 279 |
| pWISE6953 | L139F | 356 | 280 |
| pWISE6954 | L139A | 357 | 281 |
| pWISE6955 | L139I | 358 | 282 |
| pWISE6956 | L139C | 359 | 283 |
| pWISE6957 | V223C | 360 | 284 |
| pWISE6958 | V223M | 361 | 285 |
| pWISE6959 | V223L | 362 | 286 |
| pWISE6960 | V223F | 363 | 287 |
| pWISE6961 | V223Y | 364 | 288 |
| pWISE6962 | V223A | 365 | 289 |

TABLE 6b
Additional MMLV-RT(5M) Variants Identified But Not Tested.

| | Variant (Mutations | DNA SEQ | Protein SEQ |
|---|---|---|---|
| pWISE | in addition to 5M) | ID NO: | ID NO: |
| pWISE6963 | T287H | 366 | 290 |
| pWISE7086 | T287N | 367 | 291 |
| pWISE7087 | T287I | 368 | 292 |
| pWISE7088 | T287A | 369 | 293 |
| pWISE7089 | T287G | 370 | 294 |
| pWISE7090 | T287D | 371 | 295 |
| pWISE7091 | F309A | 372 | 296 |
| pWISE7092 | F309N | 373 | 297 |
| pWISE7093 | F309S | 374 | 298 |
| pWISE7094 | F309C | 375 | 299 |
| pWISE7095 | F309G | 376 | 300 |
| pWISE7096 | D524N | 377 | 301 |

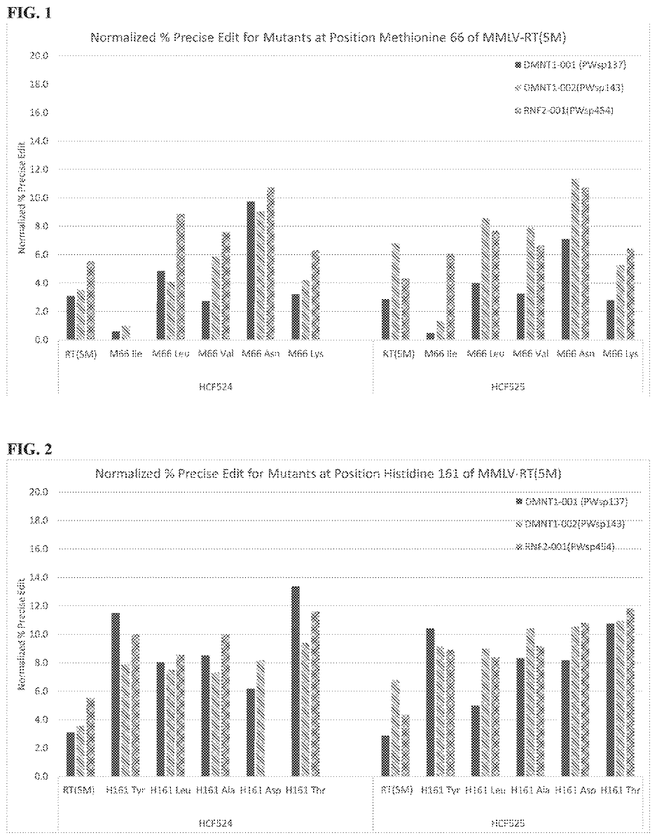

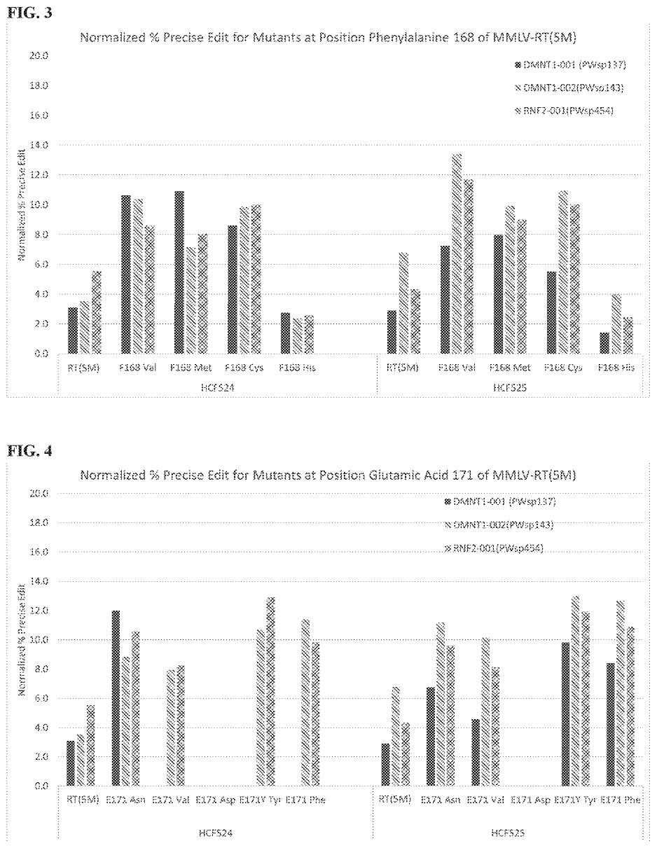

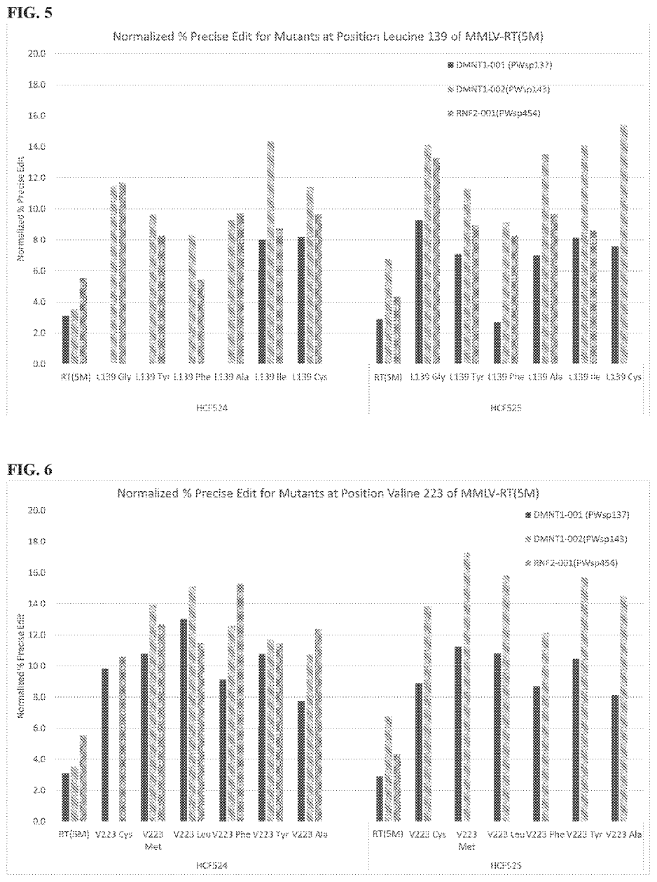

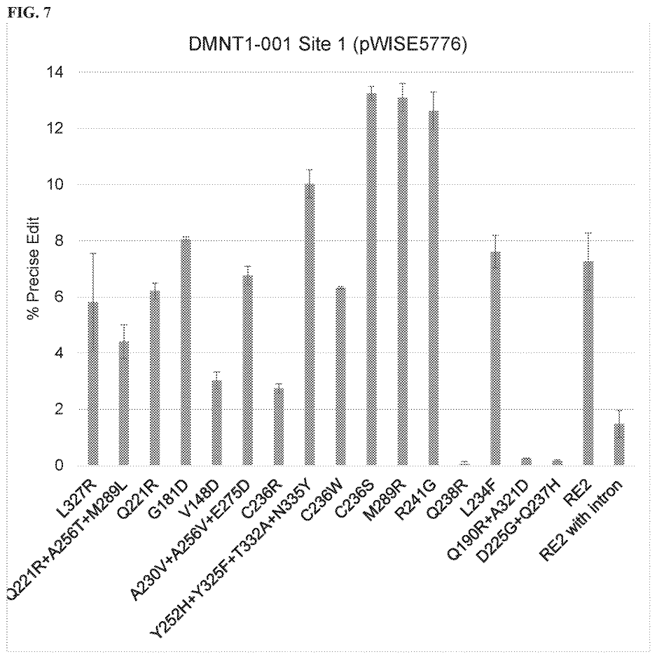

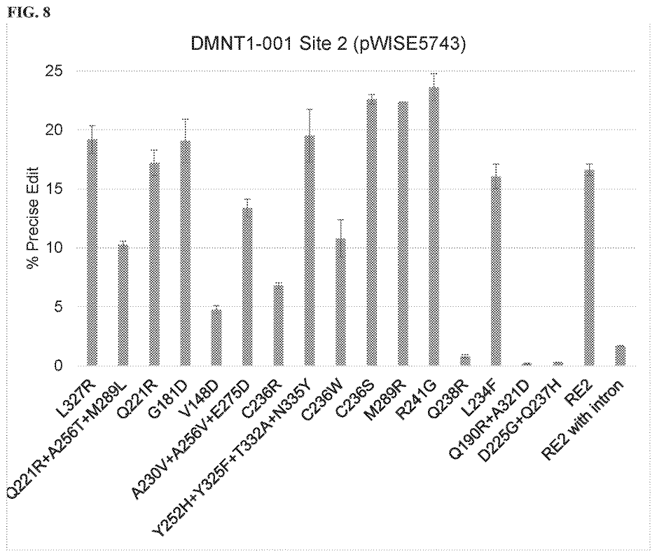

HEK293T cells were seeded into 48-well collagen-coated plates (Corning) in the absence of an antibiotic using Dulbecco's Modified Eagle Medium (DMEM) media. At 70-80% confluency, cells were transfected with 1.5 μL of LTX (ThermoFisher Scientific) using 500 ng of the control or putative RT protein plasmid and 500 ng of guide RNA plasmid according to manufacturer's protocol. After 3 days, the cells were lysed with a crude extraction method using SDS buffer. The MMLV-RT (5M) (RT (5M)) control (pWISE1654, SEQ ID NO: 258) or RT variant was scored based on the precise base pair editing and indel placement percentage at two target sites in the DNMT1 locus using PWsp137 and PWsp143 as the stagRNA guide nucleic acids and one target site in the RNF2 locus using PWsp454 as the stagRNA guide nucleic acid. As seen in FIGS. 1-6, certain variant reverse transcriptases showed activity in precise editing, HCF24 and HCF25 indicate technical replicates of this experiment.

Specific variants such as M66N, F168V, F168M and F168C showed improved editing compared to the control, and certain positions such as H161, E171, L139 and V223 showed improved activity with any of the amino acid changes.

### Example 3: Stacking of Mutations

Reverse transcriptase mutations identified as described in Example 2 were stacked together in varying combinations and evaluated for editing efficiency in human HEK293T cells. The stacked mutants contained anywhere between 2-5 amino acid mutations in the reverse transcriptase. Table 7 shows the stacked mutants used in this experiment.

TABLE 7
MMLV-RT(5M) Stacked Mutants Tested
in Human Cells (HEK293T)

| | Variant (Mutations | DNA SEQ | Protein SEQ |
|---|---|---|---|
| pWISE | in addition to 5M) | ID NO: | ID NO: |
| pWISE8357 | M66N + L139G + | 378 | 302 |
| | V223L | | |
| pWISE8358 | H161T + L139G + | 379 | 303 |
| | V223L | | |
| pWISE8359 | E171Y + L139G + | 380 | 304 |
| | V223L | | |
| pWISE8360 | M66N + H161T | 381 | 305 |
| pWISE8361 | M66N + E171Y | 382 | 306 |
| pWISE8362 | H161T + E171Y | 383 | 307 |
| pWISE8363 | M66N + H161T + | 384 | 308 |
| | E171Y | | |
| pWISE8364 | L139G + V223L | 385 | 309 |
| pWISE8365 | H161T + L139G | 386 | 310 |
| pWISE8366 | H161T + V223L | 387 | 311 |
| pWISE8367 | M66N + H161T + | 388 | 312 |
| | E171Y + L139G + | | |
| | V223L | | |
| pWISE8368 | H161T + E171Y + | 389 | 313 |
| | L139G + V223L | | |
| pWISE8369 | H161Y + L139I + | 390 | 314 |
| | V223L | | |
| pWISE8370 | H161Y + L139I + | 391 | 315 |
| | V223M | | |
| pWISE8371 | H161Y + L139I | 392 | 316 |
| pWISE8372 | L139I + V223M | 393 | 317 |

A precise editing experiment as described in Example 2 was run on each of the stacked mutants in Table 7 and the results demonstrated that the mutations could be stacked together in a range of combinations without losing significant editing efficiency.

### Example 4: Additional Variants and Combinations

Additional MMLV-RT (5M) mutants were generated from the amino acid positions in Table 1. The variants are shown below in Table 8. For the purpose of increasing bacterial cloning efficiency, the reverse transcriptase variants shown in Table 8 also included an intron (SEQ ID NO:512) inserted at position 561 of the DNA or coding sequence of the variant reverse transcriptase. Including an intron increased cloning efficiency in bacteria by preventing the translation of the coding sequence into a functional reverse transcriptase enzyme in the bacterial cell.

TABLE 8
Identified MMLV-RT(5M) Error-Prone PCR Mutants

| | Variant (Mutations | DNA SEQ | Protein SEQ |
|---|---|---|---|
| pWISE | in addition to 5M) | ID NO: | ID NO: |
| pWISE8894 | L327R | 394 | 318 |
| pWISE8895 | Q221R + A256T + | 395 | 319 |
| | M289L | | |
| pWISE8896 | Q221R | 396 | 320 |
| pWISE8897 | G181D | 397 | 321 |
| pWISE8898 | V148D | 398 | 322 |
| pWISE8899 | A230V + A256V + | 399 | 323 |
| | E275D | | |
| pWISE8900 | C236R | 400 | 324 |
| pWISE8901 | Y252H + Y325F + | 401 | 325 |
| | T332A + N335Y | | |
| pWISE8902 | C236W | 402 | 326 |
| pWISE8903 | C236S | 403 | 327 |
| pWISE8904 | M289R | 404 | 328 |
| pWISE8905 | R241G | 405 | 329 |
| pWISE8906 | Q238R | 406 | 330 |
| pWISE8907 | L234F | 407 | 331 |
| pWISE8908 | Q190R + A321D | 408 | 332 |
| pWISE8909 | D225G + Q237H | 409 | 333 |

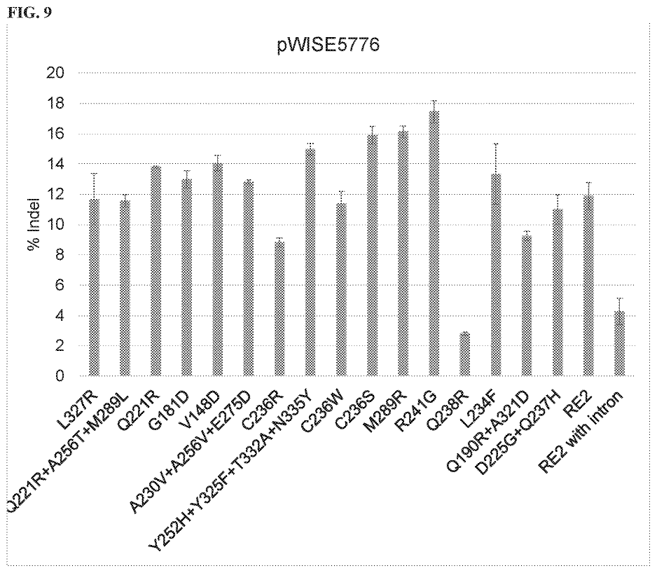

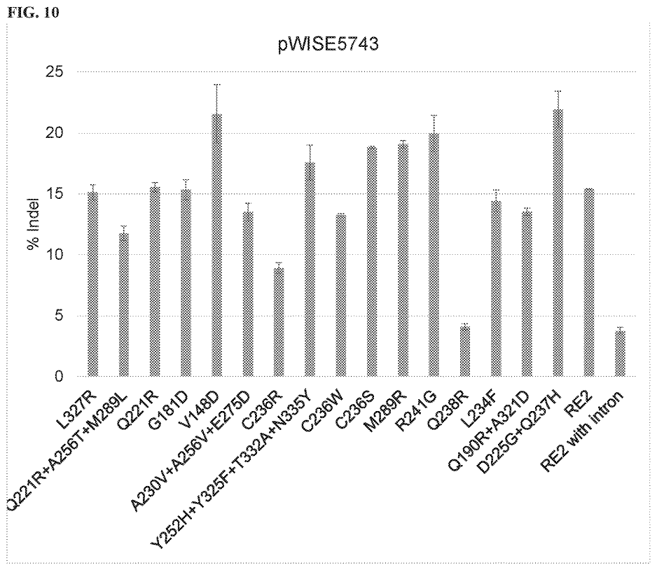

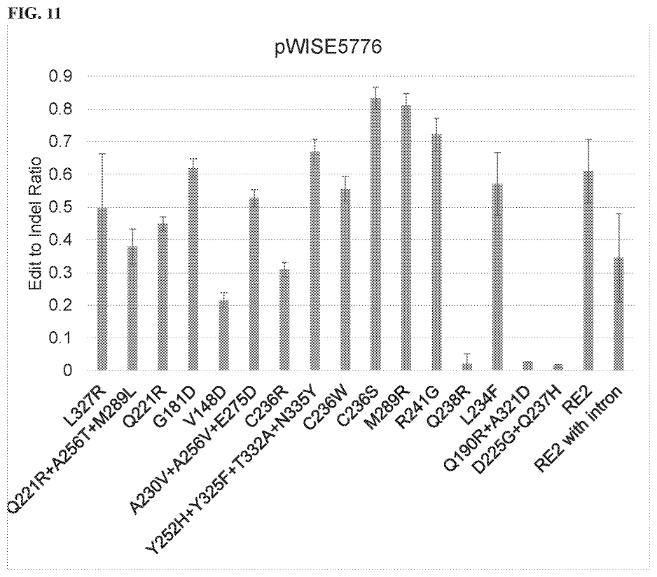

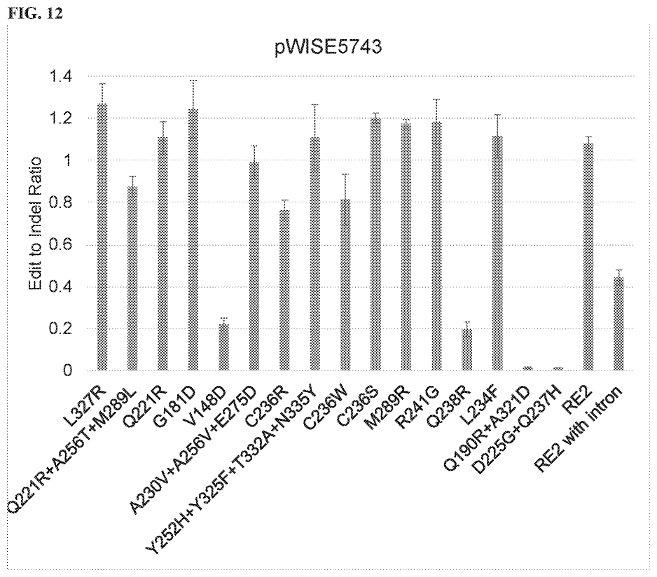

A precise editing experiment in HEK293T cells, as described in Example 3, was run on each of the mutants in Table 8 which includes single amino acid variants as well as combinations of up to 4 amino acid variants. The data is shown in FIGS. 7-12 with each figure representing editing at a different target site. A CRISPR REDRAW editor (RE2) was used as a control (pWISE6099, SEQ ID NO:513). As shown in FIGS. 7-12, the variants L327R; Q221R; G181D; V148D; A230V±A256V±E275D; C236R; Y252H±Y325F±T332A±N335Y; C236S; M289R; R241G; L234F; and D225G±Q237H showed an improvement in editing as compared to the control.

### Example 5: Precise Editing and Deletion Editing Activity

MMLV-RT (5M) mutants as listed in Table 9 were tested for their performance in precise editing and deletion editing as compared to a control.

TABLE 9
MMLV-RT(5M) Mutants Tested in Human Cells (HEK293T).

| | Variant (Mutations | DNA SEQ | Protein SEQ |
|---|---|---|---|
| pWISE | in addition to 5M) | ID NO: | ID NO: |
| pWISE6932 | M66I | 335 | 259 |
| pWISE6933 | M66L | 336 | 260 |
| pWISE6934 | M66V | 337 | 261 |
| pWISE6935 | M66N | 338 | 262 |
| pWISE6936 | M66K | 339 | 263 |
| pWISE6937 | H161Y | 340 | 264 |
| pWISE6938 | H161L | 341 | 265 |
| pWISE6939 | H161A | 342 | 266 |
| pWISE6940 | H161D | 343 | 267 |
| pWISE6941 | H161T | 344 | 268 |
| pWISE6942 | F168V | 345 | 269 |
| pWISE6943 | F168M | 346 | 270 |
| pWISE6944 | F168C | 347 | 271 |
| pWISE6945 | F168H | 348 | 272 |
| pWISE6946 | E171N | 349 | 273 |
| pWISE6947 | E171V | 350 | 274 |
| pWISE6948 | E171D | 351 | 275 |
| pWISE6949 | E171Y | 352 | 276 |
| pWISE6950 | E171F | 353 | 277 |
| pWISE6951 | L139G | 354 | 278 |
| pWISE6952 | L139Y | 355 | 279 |
| pWISE6953 | L139F | 356 | 280 |
| pWISE6954 | L139A | 357 | 281 |
| pWISE6955 | L139I | 358 | 282 |
| pWISE6956 | L139C | 359 | 283 |
| pWISE6957 | V223C | 360 | 284 |
| pWISE6958 | V223M | 361 | 285 |
| pWISE6959 | V223L | 362 | 286 |
| pWISE6960 | V223F | 363 | 287 |
| pWISE6961 | V223Y | 364 | 288 |
| pWISE6962 | V223A | 365 | 289 |
| pWISE6963 | T287H | 366 | 290 |

A precise editing experiment in HEK293T cells, as described in Example 2, was run on each of the mutants in Table 9. A CRISPR REDRAW editor (RE2) (pWISE6099, SEQ ID NO: 513) and LbCas12a (pWISE121, SEQ ID NO:59) were each used as a control. RE2 was used as a comparison to the mutants, and LbCas12a was used as a quality control for transfection of plasmids into the HEK293T cells and to confirm that editing was occurring at the expected levels.

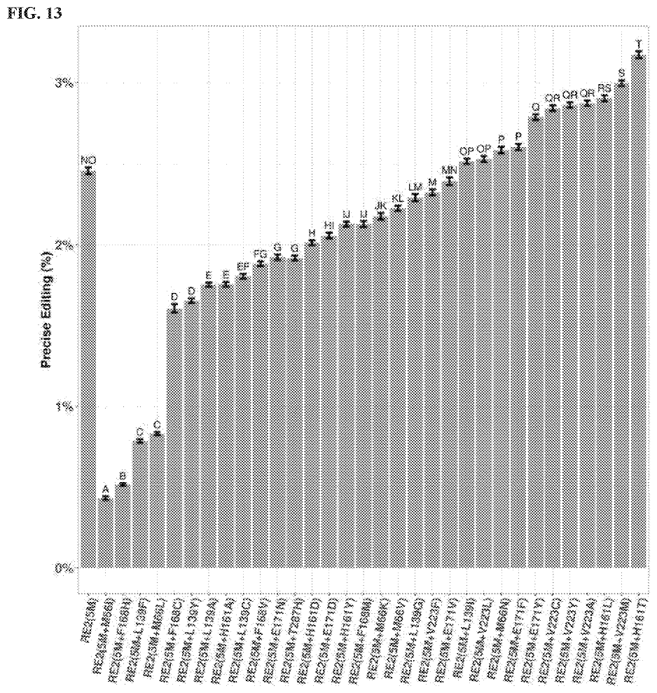

As shown in FIG. 13, the results demonstrated that 9 out of the 32 mutants showed statistically superior precise editing power as compared to the RE2 control. The mutants H161T (3.17%), V223M (3.00%), H161L (2.90%), V223A (2.87%), V223Y (2.86%), V223C (2.84%), E171Y (2.79%), E171F (2.61%), M66N (2.59%), showed the best precise editing power. The percentages indicate the percent of reads showing precise editing for each mutant.

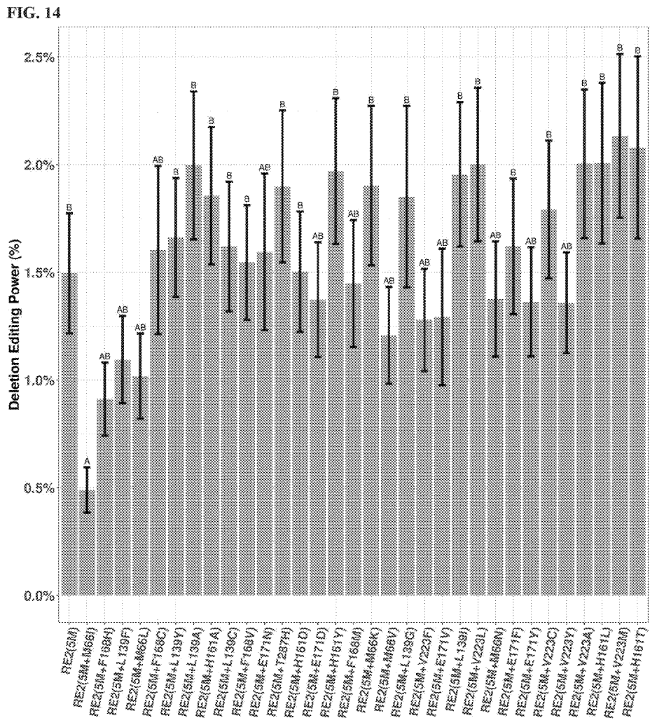

FIG. 14 shows the results from the evaluation of the rate of deletion edits, which informs whether the mutants increase double stranded breaks over improved utilization of the reverse transcriptase. Mutations with similar deletion rates as compared to the RE2 control indicate the desired outcome of improved utilization of the reverse transcriptase. The deletion power is the sum of the edit strength for all deletion edits. Each of the mutants and controls used the same stagRNAS (“RTT guides”). The test mutant M66I provided statistically lower deletion edit power as compared to the RE2 control (FIG. 14). There was no statistical evidence of a difference in the deletion edit power for the other mutants as compared to the RE2 control (FIG. 14). This indicates that the 9 top mutants improved precise editing efficiency without a general increase in the deletion rates as compared to the RE2 control.

An analysis of the position of the mutation was included that combined samples with a mutation at the same location in the enzyme. The mutations at positions H161 (2.4%), E171 (2.4%) and V223 (2.8%) provided statistically the best precise editing power (>2.3%) followed by mutations at T287 (1.9%), L139 (1.8%), M66 (1.7%), and F268 (1.5%) with the latter two positions being the lowest performing and below the RE2 control. The top 2 mutation positions (H161 and V223) contained all 5 of the top performing mutants from the per-mutant model;

however, the exact mutation at each position matters, as V223F and H161D showed no statistical improvement over the RE2 control.

### Example 6: Additional Variants

Additional MMLV-RT (5M) mutants were tested and are listed in Table 10.

TABLE 10
MMLV-RT(5M) Variants Tested in Human Cells (HEK293T).

| | Variant (Mutations | Protein SEQ |
|---|---|---|
| pWISE | in addition to 5M) | ID NO: |
| pWISE10324 | Y252H + Y325F + | 325 |
| | T332A + N335Y | |
| pWISE10325 | C236S | 327 |
| pWISE10326 | M289R | 328 |
| pWISE10327 | R241G | 329 |
| pWISE10328 | Y252H + Y325F + | 519 |
| | T332A + N335Y + | |
| | C236S | |
| pWISE10329 | Y252H + Y325F + | 518 |
| | T332A + N335Y + | |
| | M289R | |
| pWISE10330 | Y252H + Y325F + | 514 |
| | T332A + N335Y + | |
| | R241G | |
| pWISE10331 | C236S + M289R | 517 |
| pWISE10332 | C236S + R241G | 516 |
| pWISE10333 | M289R + R241G | 523 |
| pWISE10334 | Y252H + Y325F + | 521 |
| | T332A + N335Y + | |
| | C236S + M289R | |
| pWISE10335 | Y252H + Y325F + | 524 |
| | T332A + N335Y + | |
| | C236S + R241G | |
| pWISE10336 | Y252H + Y325F + | 520 |
| | T332A + N335Y + | |
| | M289R + R241G | |
| pWISE10337 | C236S + M289R + | 515 |
| | R241G | |
| pWISE10338 | Y252H + Y325F + | 522 |
| | T332A + N335Y + | |
| | C236S + M289R + | |
| | R241G | |

A precise editing experiment in HEK293T cells, as described in Example 2, was run on each of the mutants in Table 10. A CRISPR REDRAW editor (RE2) (pWISE6099, SEQ ID NO: 513) and LbCas12a (pWISE121, SEQ ID NO:59) were each used as a control. RE2 was used as a comparison to the mutants, and LbCas12a was used as a quality control for transfection of plasmids into the HEK293T cells and to confirm that editing was occurring at the expected levels.

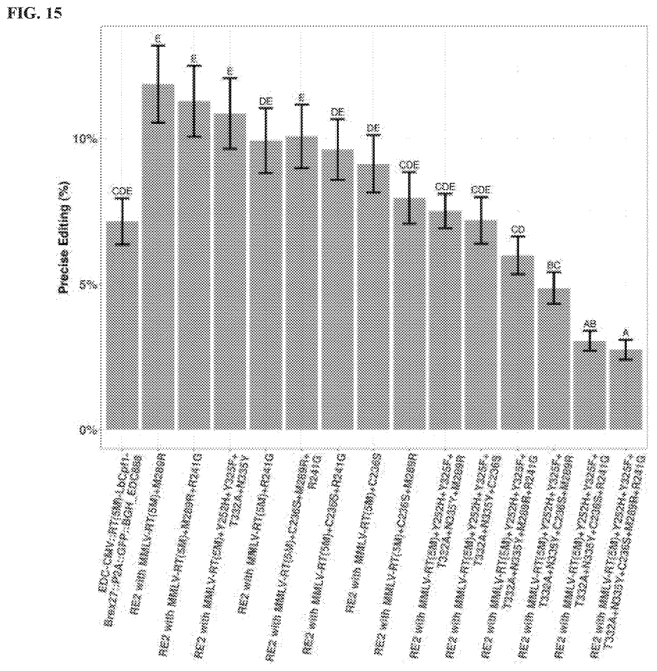

As shown in FIG. 15, the results demonstrated that the mutants comprising the following mutations: M289R; M289R+R241G; Y252H+Y325F+T332A+N335Y; R241G; C236S+M289R+R241G; C236S+R241G; and C236S had a significant increase in precise editing power as compared to the RE2 control. The highest performing mutant comprised the M289R mutation (SEQ ID NO:328), which had a marginal mean precise edit power of 11.9% compared to the control mean of 7.2%. The M289R+R241G mutant (SEQ ID NO:523), had the second highest marginal mean precise edit power of 11.3% compared to the control mean of 7.2%.

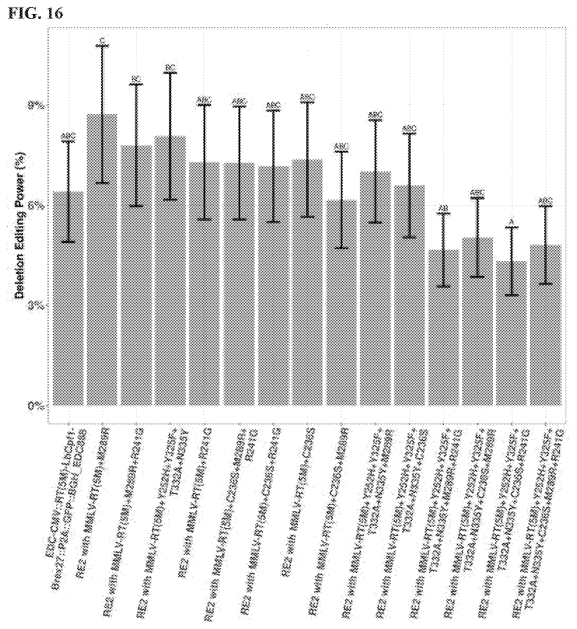

The evaluation of the rate of deletion edits informs whether the mutants increase double stranded breaks over improved utilization of the reverse transcriptase. Mutations with similar deletion rates as the RE2 control indicate the desired outcome of improved utilization of the reverse transcriptase. As shown in FIG. 16, the RE2 control had a 6.4% deletion rate and the range of the deletion rate for the mutants was 4.3% to 8.7%. All mutants had statistically equivalent deletion rates to the RE2 control. The mutants comprising M289R; M289R+R241G; Y252H+Y325F+T332A+N335Y; R241G; C236S+M289R+R241G; C236S+R241G; and C236S mutations had the highest deletion rates of 7.2% to 8.7% and were comparable to the order of editing observed for precise editing. However, due to the relative similarity to the control these mutations are likely improving precise editing through improved utilization of the reverse transcriptase rather than increasing double stranded breaks.

The foregoing is illustrative of the present invention, and is not to be construed as limiting thereof. The invention is defined by the following claims, with equivalents of the claims to be included therein.

1. A polypeptide comprising an amino acid sequence having at least 70%, 75%, 80%, 85%, 90%, 95%, 96%, 97%, 98%, 99%, or more sequence identity to SEQ ID NO:258 when the amino acid sequence is optimally aligned thereto, wherein the amino acid sequence, when optimally aligned to SEQ ID NO:258, has:
an alanine (A), asparagine (N), aspartic acid (D), arginine (R), cysteine (C), glutamic acid (E), glutamine (Q), glycine (G), histidine (H), isoleucine (I), lysine (K), methionine (M), phenylalanine (F), proline (P), serine(S), threonine (T), tryptophan (W), tyrosine (Y), or valine (V) at an amino acid residue corresponding to amino acid residue 139 of SEQ ID NO:258;
a D at an amino acid residue corresponding to amino acid residue 148 of SEQ ID NO:258;
an A, N, D, R, C, E, Q, G, I, leucine (L), K, M, F, P, S, T, W, Y, or V at an amino acid residue corresponding to amino acid residue 161 of SEQ ID NO:258;
a C, M, or V at an amino acid residue corresponding to amino acid residue 168 of SEQ ID NO: 258;
an A, N, D, R, C, Q, G, H, I, L, K, M, F, P, S, T, W, Y, or V at an amino acid residue corresponding to amino acid residue 171 of SEQ ID NO:258;
a D at an amino acid residue corresponding to amino acid residue 181 of SEQ ID NO:258;
an R at an amino acid residue corresponding to amino acid residue 221 of SEQ ID NO:258;
a G at an amino acid residue corresponding to amino acid residue 225 of SEQ ID NO:258;
a V at an amino acid residue corresponding to amino acid residue 230 of SEQ ID NO:258;
an F at an amino acid residue corresponding to amino acid residue 234 of SEQ ID NO:258;
an R or S at an amino acid residue corresponding to amino acid residue 236 of SEQ ID NO: 258;
an H at an amino acid residue corresponding to amino acid residue 237 of SEQ ID NO:258;
a G at an amino acid residue corresponding to amino acid residue 241 of SEQ ID NO:258;
an H at an amino acid residue corresponding to amino acid residue 252 of SEQ ID NO:258;
a V at an amino acid residue corresponding to amino acid residue 256 of SEQ ID NO:258;
a D at an amino acid residue corresponding to amino acid residue 275 of SEQ ID NO:258;
an H at an amino acid residue corresponding to amino acid residue 287 of SEQ ID NO:258;
an R at an amino acid residue corresponding to amino acid residue 289 of SEQ ID NO:258;
an F at an amino acid residue corresponding to amino acid residue 325 of SEQ ID NO:258;
an R at an amino acid residue corresponding to amino acid residue 327 of SEQ ID NO:258;
an A at an amino acid residue corresponding to amino acid residue 332 of SEQ ID NO:258; and/or
a Y at an amino acid residue corresponding to amino acid residue 335 of SEQ ID NO:258,
optionally wherein the polypeptide has an N at an amino acid residue corresponding to amino acid residue 200 of SEQ ID NO:258, a K at an amino acid residue corresponding to amino acid residue 306 of SEQ ID NO:258, an F at an amino acid residue corresponding to amino acid residue 313 of SEQ ID NO:258, a P at an amino acid residue corresponding to amino acid residue 330 of SEQ ID NO:258, and/or a W at an amino acid residue corresponding to amino acid residue 603 of SEQ ID NO:258.

2. The polypeptide of claim 1, wherein the amino acid sequence, when optimally aligned to SEQ ID NO:258, has:
a D at an amino acid residue corresponding to amino acid residue 148 of SEQ ID NO:258;
a D at an amino acid residue corresponding to amino acid residue 181 of SEQ ID NO:258;
an R at an amino acid residue corresponding to amino acid residue 221 of SEQ ID NO:258;
an F at an amino acid residue corresponding to amino acid residue 234 of SEQ ID NO:258;
an R or S at an amino acid residue corresponding to amino acid residue 236 of SEQ ID NO: 258;
a G at an amino acid residue corresponding to amino acid residue 241 of SEQ ID NO:258;
an R at an amino acid residue corresponding to amino acid residue 289 of SEQ ID NO:258; and/or
an R at an amino acid residue corresponding to amino acid residue 327 of SEQ ID NO:258.

3. The polypeptide of claim 1, wherein the amino acid sequence, when optimally aligned to SEQ ID NO:258, has:
a G at an amino acid residue corresponding to amino acid residue 225 of SEQ ID NO:258 and an H at an amino acid residue corresponding to amino acid residue 237 of SEQ ID NO:258;
a V at an amino acid residue corresponding to amino acid residue 230 of SEQ ID NO:258, a V at an amino acid residue corresponding to amino acid residue 256 of SEQ ID NO:258, and a D at an amino acid residue corresponding to amino acid residue 275 of SEQ ID NO:258; and/or
an H at an amino acid residue corresponding to amino acid residue 252 of SEQ ID NO:258, an F at an amino acid residue corresponding to amino acid residue 325 of SEQ ID NO:258, an A at an amino acid residue corresponding to amino acid residue 332 of SEQ ID NO:258, and a Y at an amino acid residue corresponding to amino acid residue 335 of SEQ ID NO:258.

4. The polypeptide of claim 1, wherein the amino acid sequence, when optimally aligned to SEQ ID NO:258, has:
a C, M, or V at an amino acid residue corresponding to amino acid residue 168 of SEQ ID NO: 258.

5. The polypeptide of claim 1, wherein the amino acid sequence, when optimally aligned to SEQ ID NO:258, has:
a C, M, or V at an amino acid residue corresponding to amino acid residue 168 of SEQ ID NO: 258
an A, N, D, R, C, E, Q, G, H, I, K, M, F, P, S, T, W, Y, or V at an amino acid residue corresponding to amino acid residue 139 of SEQ ID NO:258;
an A, N, D, R, C, E, Q, G, I, L, K, M, F, P, S, T, W, Y, or V at an amino acid residue corresponding to amino acid residue 161 of SEQ ID NO:258; and/or
an A, N, D, R, C, Q, G, H, I, L, K, M, F, P, S, T, W, Y, or V at an amino acid residue corresponding to amino acid residue 171 of SEQ ID NO:258.

6. The polypeptide of claim 1, wherein the amino acid sequence, when optimally aligned to SEQ ID NO:258, further comprises:
an A, N, D, R, C, E, Q, G, H, I, L, K, M, F, P, S, T, W, or Y at an amino acid residue corresponding to amino acid residue 223 of SEQ ID NO:258.

7. The polypeptide of claim 1, wherein the amino acid sequence has at least 70% sequence identity to the amino acid sequence of one of SEQ ID NOs: 264-271, 273-283, 303-318, 320-325, 327-329, 331, 333, and 514-524.

8. The polypeptide of claim 1, wherein the polypeptide has activity as an RNA-dependent DNA polymerase and/or wherein the polypeptide generates the DNA by polymerizing the DNA from one end of a DNA and/or RNA primer.

9. The polypeptide of claim 1, wherein the polypeptide generates the DNA from the RNA at a temperature of about 40° C. or less.

10. The polypeptide of claim 1, wherein the polypeptide has a processivity of at least about 100, 250, 500, 1000, 1500, or 2000 nucleotides.

11-13. (canceled)

14. The polypeptide of claim 1, wherein the polypeptide is devoid of at least a portion of an RNAseH domain.

15. The polypeptide of claim 1, wherein the polypeptide generates the DNA from the RNA in two or more different species.

16. The polypeptide of claim 1, wherein the polypeptide has a processivity of at least 100 nucleotides at a temperature in a range of about 25° C. to about 80° C. in two or more different species.

17-20. (canceled)

21. The polypeptide of claim 1, further comprising a CRISPR-Cas effector polypeptide that is fused to the polypeptide.

22-23. (canceled)

24. A nucleic acid encoding the polypeptide of claim 1.

25. A complex comprising:
a CRISPR-Cas effector protein;
the polypeptide of claim 1; and
an extended guide nucleic acid.

26-34. (canceled)

35. An expression cassette codon optimized for expression in an organism, comprising:
a polynucleotide encoding a promoter sequence, and
a polynucleotide encoding the polypeptide of claim 1,
optionally wherein the polynucleotide encoding the polypeptide is codon-optimized for expression in the organism.

36-37. (canceled)

38. A method of modifying a target nucleic acid in a cell, the method comprising:
introducing the expression cassette of claim 35 into the cell, thereby modifying the target nucleic acid in the cell.

39-42. (canceled)

43. A method for performing reverse transcription, the method comprising:
contacting a target nucleic acid with the polypeptide of claim 1,
wherein the polypeptide reverse transcribes the target nucleic acid to provide a DNA.

44. A method of modifying a target nucleic acid, the method comprising:
contacting the target nucleic acid with
a CRISPR-Cas effector protein;
the polypeptide of claim 1; and
an extended guide nucleic acid, thereby modifying the target nucleic acid.

45-67. (canceled)
